(19) United States
(12) Patent Application Publication
(10) Pub. No.: US 2008/0269282 A1
(43) Pub. Date: Oct. 30, 2008

(54) Compounds for Inhibiting Copper-Containing Amine Oxidases and Uses Thereof

(75) Inventors: Luc Marti Clauzel, Barcelona (ES); Silvia Garcia Vicente, Sant Feliu de Llobregat (ES); Francesc Yraola Font, Barcelona (ES); Miriam Royo Exposito, Barcelona (ES); Fernando Albericio Palomera, Barcelona (ES); Antonio Zorzano Olarte, Barcelona (ES)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: GENMEDICA THERAPEUTICS SL, Barcelona (ES)

(21) Appl. No.: 11/573,089

(22) PCT Filed: Aug. 2, 2005
(86) PCT No.: PCT/EP05/53778

§ 371 Date: May 9, 2008

Related U.S. Application Data
(60) Provisional application No. 60/598,010, filed on Aug. 2, 2004.

(51) Int. Cl. A61K 31/165; C07C 233/01; C07C 237/00; C07D 209/04; A61K 31/404; A61K 31/47; A61P 27/06; A61P 3/00; A61P 17/00; A61P 37/00; A61P 19/02; A61P 29/00; C07C 381/00; C07D 215/00

(52) U.S. Cl. 514/311; 564/180; 564/155; 548/495; 564/162; 546/168; 514/617; 514/616; 514/419; 514/618

(57) ABSTRACT

This invention is directed to inhibitors of copper-containing amine oxidases (E.C.1.4.3.6) including semicarbazide-sensitive amine oxidase (SSAO; also known as vascular adhesion protein-1, VAP-I), and their therapeutic use in inflammatory diseases, diabetes and its associated complications, atherosclerosis, neurodegenerative diseases, obesity, hypertension and cancer.

This applications claims priority to U.S. provisional application Ser. No. 60/598,010, filed Aug. 2, 2004, the disclosure of which is explicitly incorporated by reference herein.

### BACKGROUND OF THE INVENTION

1. Field of the Invention

The present invention relates to inhibitors of copper-containing amine oxidases (E.C. 1.4.3.6). Specifically, the invention provides inhibitors of semicarbazide-sensitive amine oxidase (SSAO; also known as Vascular adhesion protein-1 VAP-1). The invention provides methods for using such compounds as therapeutic agents for treating inflammatory diseases, diabetes and its associated complications, atherosclerosis, neurodegenerative diseases, obesity, hypertension and cancer.

2. Background of the Related Art

Semicarbazide-sensitive amine oxidase (SSAO)/Vascular Adhesion Protein-1 (VAP-1) is a bifunctional membrane protein. One function of this protein is as a copper-containing ectoenzyme with amine oxidase activity that can be inhibited by carbonyl-reactive compounds such as semicarbazide (Lyles, 1996, Int. J. Biochem. Cell Biol. 28:259-274). SSAO oxidizes a primary amine into the corresponding aldehyde with production of hydrogen peroxide and ammonia according to the following reaction:

R—CH₂—NH₂+O₂→R—CHO+H₂O₂+NH₃

SSAO/VAP-1 is also an adhesion molecule implicated in inflammation processes (Bono et al., 1999, Amer J Pathol 155:1613-1624; Salmi & Jalkanen, 1992, Science 257:1407-1409; Smith et al., 1998, J. Exp. Med. 188:17-27)

SSAO/VAP-1 is expressed in a variety of tissues, including endothelial cells, lung, smooth muscle cells, and (under normal conditions, highly expressed) in adipose tissue cells. SSAO/VAP-1 is not expressed in 3T3-L1 fibroblasts, but is induced during adipogenesis (Fontana et al., 2001, Biochem. J 356:769-777; Moldes et al., 1999, J. Biol. Chem. 274:9515-9523). This suggests that SSAO/VAP-1 is a member of the adipogenic gene program and, in addition, that SSAO/VAP-1 may contribute to the acquisition of some final characteristics of fully differentiated adipose cells.

SSAO substrates are known to strongly stimulate glucose transport and recruitment of GLUT4 to the cell surface in isolated rat adipocytes or 3T3-L1 adipocytes (Enrique-Tarancon et al., 1998, J. Biol. Chem. 273:8025-8032; Enrique-Tarancon et al., 2000, Biochem. J. 350:171-180; Fontana et al., 2001, Biochem. J 356:769-777; Marti et al., 1998, J Pharmacol. Exp. Ther. 285:342-349). Stimulation of glucose transport by SSAO substrates has also been demonstrated in isolated human adipocytes (Morin et al., 2001, J Pharmacol. Exp. Ther. 297:563-572).

The identity of SSAO and VAP-1 has more recently been established (Bono et al., 1999, Amer J Pathol 155:1613-1624; Smith et al., 1998, J Exp. Med. 188:17-27) VAP-1, first disclosed in Salmi et al. in 1992 (Salmi & Jalkanen, 1992, Science 257:1407-1409) is upregulated (i.e., its expression increases) on the vascular endothelium at inflammation sites, and mediates a multistep adhesive process leading to the transmigration of leukocytes from the circulation into inflamed tissues. Lymphocyte adhesion to endothelial cells is mediated by SSAO/VAP-1 in a sialic acid-dependent manner (Bono et al., 1998, J Immunol. 160:5563-5571) and more recently it has been shown that the SSAO amine oxidase activity of VAP-1/SSAO also participates to the adhesive function of VAP-1 (Salmi et al., 2001, Immunity. 14:265-276).

VAP-1/SSAO has been implicated in a variety of inflammatory responses through its enzymatic activity. These include lymphocyte adhesion (Kurkijarvi et al., 1998, J Immunol. 161:1549-1557; Salmi & Jalkanen, 1992, Science 257:1407-1409; Salmi et al., 2001, Immunity. 14:265-276); and production of aldehydes like formaldehyde or methylglyoxal, putatively endogenous products of SSAO, by generating protein cross-linking and advanced glycosylation end-product (AGE) formation (Yu, 1998, J Neural Transom. Supply 52:201-21). Additionally, VAP-1/SSAO has the capacity to promote LDL oxidation in vitro (Exner et al., 2001, Cardiovasc. Res. 50:583-588) (perhaps through its copper ion), and mice overexpressing VAP-1/SSAO in endothelial cells have a propensity to atherosclerosis (Stolen et al., 2004, FASEB J. 18: 702-704).

VAP-1/SSAO has also been implicated in cardiovascular complications associated with diabetes, adipogenicity, apoptosis secondary to stroke and hypertension. The higher activity of SSAO associated with diabetes as well as a higher concentration of its putative endogenous substrates, methylamine and aminoacetone, may result in greater production of formaldehyde, methylglyoxal and hydrogen peroxide in diabetics than in normal individuals. These products are highly cytotoxic for endothelial cells, which may lead to cardiovascular complications associated to diabetes (Yu, 1998, J Neural Transm. Suppl 52:201-218).

In addition to membrane-bound adhesion molecules, soluble isoforms of SSAO/VAP-1 have been detected in blood plasma from healthy individuals (Gearing & Newman, 1993, Immunol. Today 14:506-512, 1993; Kurkijarvi et al., 1998, J Immunol. 161:1549-1557; Rothlein et al., 1991, J Immunol. 147:3788-3793). The soluble form of VAP-1/SSAO is found in healthy adult plasma at concentrations of 50-140 ng/mL, which is enhanced in inflammatory liver diseases (Kurkijarvi et al., 1998, J Immunol. 161:1549-1557), cardiovascular pathologies (Boomsma et al., 1997, Cardiovasc. Res. 33:387-391), endstage of renal disease (Kurkijarvi et al., 2001, Eur. J Immunol. 31:2876-2884), obesity (Meszaros et al., 1999, Metabolism 48:113-117; Weiss et al., 2003, Metabolism 52:688-692) type I diabetes (Hayes & Clarke, 1990, Res. Commun. Chem. Pathol Pharmacol. 69:71-83; Boomsma et al., 1995, Clin. Sci. (Lond) 88:675-679; Boomsma et al., 1999, Diabetologia 42:233-237; Meszaros et al., 1999, Metabolism 48:113-117; Salmi et al., 2002, Am J Pathol 161:2255-2262) and type II diabetes (Boomsma et al., 1999, Diabetologia 42:233-237; Garpenstrand et al., 1999, Diabet. Med. 16:514-521; Meszaros et al., 1999, Metabolism 48:113-117). The soluble form of VAP-1/SSAO enhances the binding capacity of lymphocytes to endothelial cells (Kurkijarvi et al., 1998, J Immunol. 161:1549-1557) presumably through a lymphocyte preactivation signal. In addition, SSAO aldehyde products, such as formaldehyde or methylglyoxal, may generate protein cross-linking or AGE products implicated in atherogenic lesions, retinopathy and angiopathy associated with diabetes.

Because all these events can promote inflammation and atherosclerosis (Osterud & Bjorklid, 2003, Physiol Rev. 83:1069-1112), pharmacological inhibition of VAP-1/SSAO may reduce a variety of pathologies. A need exists, therefore, for inhibitors of SSAO/VAP-1 that can be used therapeutically to alleviate the symptoms and pathology associated with expression or over-expression of SSAO/VAP-1 in a number of disease states and other disorders.

### SUMMARY OF THE INVENTION

The present invention provides SSAO/VAP-1 inhibitors having the general formula I:

R₃—(Y)ₙ—(CR₂R₂)ₘ-Z

or a pharmaceutically acceptable salt thereof, wherein
m is 0 or 1-6 (in another aspect, m is 0 or 1-4);
n is 0 or 1-6 (in another aspect, n is 0 or 1-4);
Z is CONR₁OH, COOH, B(OH)₂, SO₂NR₁OH, OR₁, SR₁, NHR₁, PO₃H, CH₂NHR₁, COR₁, CONHR₁, CHNR₁, or CNR₁NHR₁;
Y at each occurrence is independently —CO—, —CS—, —NR₂OR₂—, —NR₂—, —SR₂—, —NR₂SO₂R₂—, —COR₂—, —NR₂—C(NR₂)—NR₂—, —(C₁-C₆ alkyl)-NHC(O)—, —(C₁-C₆ alkyl)-N(C₁-C₆ alkyl)C(O)—, —NHC(O)—(C₁-C₆ alkyl)-, —N(C₁-C₆ alkyl)C(O)—(C₁-C₆ alkyl)-, —NHC(O)—, —N(C₁-C₆alkyl)C(O)—, —C(O)NH—, —C(O)—N(C₁-C₆alkyl), —SO₂NH—, —SO₂—N(C₁-C₆alkyl)-, —(C₁-C₆ alkyl)-C(O)NH—, —C₁-C₆ alkyl)-C(O)—N(C₁-C₆alkyl)-, —O—(C₁-C₆ alkyl)-NHC(O)—, or —O—(C₁-C₆ alkyl)-N(C₁-C₆ alkyl)C(O)—, wherein the alkyl portion or portions of each of the above is optionally substituted with 1, 2, or 3 groups that are independently halogen, C₁-C₄ alkoxy, amino, mono or di (C₁-C₆ alkyl)amino, OH, indolyl, pyrrolyl, pyridyl, furanyl, guanidinyl, carboxyl, or ═O;
R₁ at each occurrence is independently H, C₁-C₆ alkyl, aryl, substituted aryl, heterocycloalkyl containing at least one and no more than two heteroatoms selected from S, N, and O, or C₃-C₇ cycloalkyl, and where any member of the alkyl, aryl/or cycloalkyl group is optionally substituted with halogen, C₁-C₆ alkyl or C₁-C₆ alkoxy, aryl or substituted aryl;
R₂ at each occurrence is independently H, C₁-C₆ alkyl, carboxyl, C₁-C₆ alkoxycarbonyl, aryl, substituted aryl, C₁-C₆ alkoxy, C₁-C₆ alkoxyalkyl, C₃-C₇ cycloalkyl, C₃-C₇ cycloalkylalkyl, C₃-C₇ cycloalkylalkoxy, heteroaryl, heteroarylalkyl, heterocycloalkyl, or heterocycloalkylalkyl, where each of the above is optionally substituted with 1, 2, 3, 4, or 5 groups that are independently halogen, C₁-C₆ alkyl, C₁-C₆ alkoxy, aryl, substituted aryl, halogen, nitro, nitroso, aldehyde (CHO), carboxylic acid, —C(O)NH₂, —C(O)NH(C₁-C₆ alkyl), —C(O)N(C₁-C₆ alkyl)(C₁-C₆ alkyl), amino, NH(C₁-C₆ alkyl), N(C₁-C₆ alkyl)(C₁-C₆ alkyl), C₁-C₆ alkoxycarbonyl, sulfate, imine, hydroxyl, —SH, or nitrile; and
R₃ is aryl, C₁-C₆ alkyl, C₂-C₄ alkenyl, C₃-C₇ cycloalkyl, C₃-C₇ cycloalkyl C₁-C₆ alkyl, C₃-C₇ cycloalkyl C₁-C₆ alkoxy, heteroaryl, heterocycloalkyl, each of which is optionally substituted with 1, 2, 3, 4, or 5 groups that are independently C₁-C₆ alkyl, C₁-C₆ alkoxy, halogen, haloalkyl, haloalkoxy, nitro, amino, NH(C₁-C₆ alkyl), N(C₁-C₆ alkyl)(C₁-C₆ alkyl), CN, CO₂H, C₁-C₆ alkylthio, C₁-C₄ haloalkyl, C₁-C₄ haloalkoxy, C₁-C₆ acyloxy, aryl, heteroaryl, or hydroxyl, where the aryl and heteroaryl substituents on R₃ are further optionally substituted with one or more groups that are independently C₁-C₆ alkyl, amino, NH(C₁-C₆ alkyl), N(C₁-C₆ alkyl)(C₁-C₆ alkyl), OH, NO₂, C₁-C₆ alkoxy, halogen, arylalkoxy (in one aspect, phenylalkoxy, in another aspect, phenylC₁ alkoxy), haloalkyl (in one aspect, CF₃), haloalkoxy (in one aspect, OCF₃), thiol, or C₂-C₆ alkanoyl (in one aspect, C₂ alkanoyl).

The invention also provides methods for preparing a compound of formula I.

The invention further provides compounds of formula I prepared according to the methods of the invention.

The invention specifically provides methods for inhibiting SSAO/VAP-1 using the compounds of the invention.

The invention also provides pharmaceutical compositions comprising the SSAO/VAP-1 inhibitors of the invention and a pharmaceutically-acceptable diluent, solvent, excipient and/or adjuvant.

The invention further provides methods for treating a disease or disorder associated with SSAO/VAP-1 activity in an animal, wherein said SSAO/VAP-1 activity is inhibited in the animal, preferably by administering to the animal a compound or pharmaceutical composition of the SSAO/VAP-1 inhibitors of the invention. Preferably, the animal is a human.

Specific preferred embodiments of the present invention will become evident from the following more detailed description of certain preferred embodiments and the claims.

### DETAILED DESCRIPTION OF PREFERRED EMBODIMENTS

As indicated above, the invention provides methods of preparing compounds of formula I, which are inhibitors of copper-containing amine oxidases (E.C. 1.4.3.6) including semicarbazide-sensitive amine oxidase (SSAO; also known as Vascular adhesion protein-1, VAP-1).

In one aspect, the invention provides compounds of formula I-a, i.e., compounds of formula I, or a pharmaceutically-acceptable salt thereof, wherein
Z is CONR₁OH, COOH, NHR₁, CH₂NHR₁, CONHR₁, or CHNR₁; wherein
   R₁ at each occurrence is independently H, or C₁-C₆ alkyl, phenyl, naphthyl, binaphthyl, piperidinyl, pyrrolidinyl, piperazinyl, morpholinyl, S,S-dioxomorpholinyl, or C₃-C₇ cycloalkyl, where each of the above is optionally substituted with halogen, C₁-C₆ alkyl or C₁-C₆ alkoxy, phenyl, or naphthyl, wherein the phenyl and naphthyl groups are optionally substituted with 1, 2, 3, 4, or 5 groups that are independently halogen, C₁-C₆ alkyl, C₁-C₆ alkoxy, aryl, substituted aryl, halogen, nitro, nitroso, aldehyde, carboxylic acid, amide, amine, C₁-C₆ alkoxycarbonyl, sulfate, imine, hydroxyl or nitrile.

In another aspect, the invention provides compounds of formula I-b, i.e., compounds of formula I, or a pharmaceutically-acceptable salt thereof, wherein
Y is —CO—, —COR₂—, —(C₁-C₆ alkyl)-NEC(O)—, —(C₁-C₆ alkyl)-N(C₁-C₆ alkyl)C(O)—, —NHC(O)—, —N(C₁-C₆alkyl)C(O)—, —NHC(O)—(C₁-C₆ alkyl)-, —N(C₁-C₆ alkyl)C(O)—C₁-C₆ alkyl)-, —C(O)NH—, —C(O)—N(C₁-C₆alkyl), —SO₂NH—, —SO₂—N(C₁-C₆alkyl)-, —(C₁-C₆ alkyl)-C(O)NH—, —C₁-C₆ alkyl)-C(O)—N(C₁-C₆alkyl)-, —O—(C₁-C₆ alkyl)-NHC(O)—, or —O—(C₁-C₆ alkyl)-N(C₁-C₆ alkyl)C(O)—, wherein the alkyl portion or portions of each of the above is optionally substituted with 1, 2, or 3 groups that are independently halogen, C₁-C₄ alkoxy, amino, mono or di (C₁-C₆ alkyl)amino, OH, indolyl, pyrrolyl, pyridyl, furanyl, guanidinyl, carboxyl, or ═O, wherein
   R₂ at each occurrence is independently H, C₁-C₆ alkyl, phenyl, naphthyl, C₁-C₆ alkoxy, C₁-C₆ alkoxyalkyl, C₃-C₇ cycloalkyl, C₃-C₇ cycloalkylalkyl, C₃-C₇ cycloalkylalkoxy, pyridyl, thienyl, furanyl, imidazolyl, pyrimidyl, pyrrolyl, piperidinyl, piperazinyl, pyrrolidinyl, morpholinyl, S,S-dioxomorpholinyl, piperidinyl C₁-C₄ alkyl, piperazinyl C₁-C₄ alkyl, pyrrolidinyl C₁-C₄ alkyl, morpholinyl C₁-C₄ alkyl, S,S-dioxomorpholinyl C₁-C₄ alkyl, where each of the above is optionally substituted with 1, 2, 3, 4, or 5 groups that are independently halogen, C₁-C₆ alkyl, C₁-C₆ alkoxy, aryl, substituted aryl, halogen, nitro, nitroso, aldehyde, carboxylic acid, —C(O)NH₂, —C(O)NH(C₁-C₆ alkyl), —C(O)N(C₁-C₆ alkyl)(C₁-C₆ alkyl), amino, C₁-C₆ alkoxycarbonyl, sulfate, imine, hydroxyl or nitrile.

In still another aspect, the invention provides compounds of formula I-c, i.e., compound of formula I, or a pharmaceutically-acceptable salt thereof, wherein
R₃ is aryl, selected from phenyl, naphthyl, indanyl, and biphenyl, C₅-C₆ cycloalkyl, C₅-C₆ cycloalkyl C₁-C₆ alkyl, C₂-C₄ alkenyl, C₅-C₆ cycloalkyl C₁-C₆ alkoxy, heteroaryl, selected from pyridyl, pyrimidyl, indolyl, pyrrolyl, thienyl, furanyl, thiazolyl, pyrazolyl, and oxazolyl, heterocycloalkyl, selected from piperazinyl, piperidinyl, pyrrolidinyl, quinolinyl, isoquinolinyl, tetrahydropyranyl, morpholinyl, thiomorpholinyl, and S,S-dioxothiomorpholinyl, each of which is optionally substituted with 1, 2, 3, 4, or 5 groups that are independently C₁-C₆ alkyl, C₁-C₆ alkoxy, halogen, CF₃, OCF₃, nitro, CN, CO₂H, C₁-C₆ alkylthio, C₁-C₆ acyloxy, phenyl, pyridyl, thienyl, furanyl, pyrimidyl, or hydroxy.

In yet another aspect, the invention provides compounds of formula I-d, i.e., compounds of formula I, or a pharmaceutically-acceptable salt thereof, wherein
n is 1-4;
m is 1-4;
Z is CONR₁, OH, COOH, NHR₁, CH₂NHR₁, CONHR₁, or CHNR₁; wherein
   R₁ at each occurrence is independently H, or C₁-C₆ alkyl, phenyl, naphthyl, binaphthyl, piperidinyl, pyrrolidinyl, piperazinyl, morpholinyl, S,S-dioxomorpholinyl, or C₃-C₇ cycloalkyl, where each of the above is optionally substituted with halogen, C₁-C₆ alkyl or C₁-C₆ alkoxy, phenyl, or naphthyl, wherein the phenyl and naphthyl groups are optionally substituted with 1, 2, 3, 4, or 5 groups that are independently halogen, C₁-C₆ alkyl, C₁-C₆ alkoxy, phenyl, halogen, nitro, carboxylic acid, C(O)NH₂, C(O)NH(C₁-C₆ alkyl), C(O)N(C₁-C₆ alkyl)(C₁-C₆ alkyl), NH₂, NH(C₁-C₆ alkyl), N(C₁-C₆ alkyl)(C₁-C₆ alkyl), hydroxyl or nitrile;
Y is —CO—, —NR₂—, —COR₂—, —(C₁-C₆ alkyl)-NHC(O)—, —(C₁-C₆ alkyl)-N(C₁-C₆ alkyl)C(O)—, —NHC(O)—, —N(C₁-C₆alkyl)C(O)—, —C(O)NH—, —C(O)—N(C₁-C₆alkyl), —SO₂NH—, —SO₂—N(C₁-C₆alkyl)-, —(C₁-C₆ alkyl)-C(O)NH—, —(C₁-C₆ alkyl)-C(O)—N(C₁-C₆alkyl)-, —O—(C₁-C₆ alkyl)-NHC(O)—, or —O((C₁-C₆ alkyl)-N(C₁-C₆ alkyl)C(O)—, wherein the alkyl portion or portions of each of the above is optionally substituted with 1, 2, or 3 groups that are independently halogen, C₁-C₄ alkoxy, amino, mono or di (C₁-C₆ alkyl)amino, OH, indolyl, guanidinyl, carboxyl, or ═O; wherein
   R₂ at each occurrence is independently H, C₁-C₆ alkyl, phenyl, naphthyl, C₁-C₆ alkoxy, C₁-C₆ alkoxyalkyl, C₃-C₇ cycloalkyl, C₃-C₇ cycloalkylalkyl, C₃-C₇ cycloalkylalkoxy, pyridyl, thienyl, furanyl, imidazolyl, pyrimidyl, pyrrolyl, piperidinyl, piperazinyl, pyrrolidinyl, morpholinyl, S,S-dioxomorpholinyl, piperidinyl C₁-C₄ alkyl, piperazinyl C₁-C₄ alkyl, pyrrolidinyl C₁-C₄ alkyl, morpholinyl C₁-C₄ alkyl, S,S-dioxomorpholinyl C₁-C₄ alkyl, where each of the above is optionally substituted with 1, 2, 3, 4, or 5 groups that are independently halogen, C₁-C₆ alkyl, C₁-C₆ alkoxy, aryl, substituted aryl, halogen, nitro, nitroso, aldehyde, carboxylic acid, C(O)NH₂, C(O)NH(C₁-C₆ alkyl), C(O)N(C₁-C₆ alkyl)(C₁-C₆ alkyl), NH₂, NH(C₁-C₆ alkyl), N(C₁-C₆ alkyl)(C₁-C₆ alkyl), C₁-C₆ alkoxycarbonyl, sulfate, imine, hydroxyl, —SH, or nitrile; and
R₃ is aryl selected from phenyl, naphthyl, indanyl, and biphenyl, C₅-C₆ cycloalkyl, heteroaryl selected from pyridyl, pyrimidyl, indolyl, pyrrolyl, thienyl, furanyl, thiazolyl, pyrazolyl, and oxazolyl, heterocycloalkyl selected from piperazinyl, piperidinyl, pyrrolidinyl, quinolinyl, tetrahydropyranyl, morpholinyl, thiomorpholinyl, and S,S-dioxothiomorpholinyl, each of which is optionally substituted with 1, 2, 3, 4, or 5 groups that are independently C₁-C₆ alkyl, C₁-C₆ alkoxy, halogen, CF₃, OCF₃, nitro, CN, CO₂H, C₁-C₆ alkylthio, C₁-C₆ acyloxy, phenyl, pyridyl, thienyl, furanyl, pyrimidyl, or hydroxy.

In another aspect, the invention provides compounds of formula I-e, i.e., compounds of formula I-d, or a pharmaceutically-acceptable salt thereof, wherein
Z is CONR₁OH, or NHR₁, wherein
R₁ at each occurrence is independently H, or C₁-C₆ alkyl, wherein the alkyl group is optionally substituted with halogen, or C₁-C₆ alkoxy, or phenyl, wherein the phenyl group is optionally substituted with 1, 2, 3, 4, or 5 groups that are independently halogen, C₁-C₆ alkyl, C₁-C₆ alkoxy, phenyl, halogen, nitro, carboxylic acid, C(O)NH₂, C(O)NH(C₁-C₆ alkyl), C(O)N(C₁-C₆ alkyl)(C₁-C₆ alkyl), NH₂, NH(C₁-C₆ alkyl), N(C₁-C₆ alkyl)(C₁-C₆ alkyl), hydroxyl.

In another aspect, the invention provides compounds of formula I-f, i.e., compounds of formula I-c or I-d, or a pharmaceutically-acceptable salt thereof, wherein Z is CONR₁OH, and R₁ is H, or C₁-C₆ alkyl.

In yet another aspect, the invention provides compounds of formula I-g, i.e., compounds of formula I-c or I-d, or a pharmaceutically-acceptable salt thereof, wherein R₁ is H.

In still another aspect, the invention provides compounds of formula I-h, i.e., compounds of formula I-c or I-d, or a pharmaceutically-acceptable salt thereof, wherein R₁ is C₁-C₆ alkyl.

In still yet another aspect, the invention provides compounds of formula I-i, i.e., compounds of formula I-c or I-d, or a pharmaceutically-acceptable salt thereof, wherein Z is CONHR₁.

In yet still another aspect, the invention provides compounds of formula I-j, i.e., compounds of formula I-c or I-d, wherein R₂ is independently H, or C₁-C₆ alkyl, where the alkyl group is optionally substituted with one or two groups that are independently halogen, C₁-C₄ alkoxy, phenyl, naphthyl, nitro, CHO, carboxyl, C(O)NH₂, C(O)N—H(C₁-C₆ alkyl), C(O)N(C₁-C₆ alkyl)(C₁-C₆ alkyl), NH₂, NH(C₁-C₆ alkyl), N(C₁-C₆ alkyl)(C₁-C₆ alkyl), hydroxyl, C₁-C₆ alkoxycarbonyl, or nitrile.

In another aspect, the invention provides compounds of formula I-k, i.e., compounds of formula I-j, wherein one R₂ is H and the other is H or C₁-C₆ alkyl, where the alkyl group is optionally substituted with OH, NH₂, or SH.

In another aspect, the invention provides compounds of formula I-1, i.e., compounds of formula I, I-a, I-b, I-c, I-d, I-e, I-f, I-g, I-h, I-l, I-j, or I-k, wherein Y is —NR₂—, —(C₁-C₆ alkyl)-NHC(O)—, —(C₁-C₆ alkyl)-N(C₁-C₆ alkyl)C(O)—, —NHC(O)—, —N(C₁-C₆alkyl)C(O)—, —C(O)NH—, —C(O)—N(C₁-C₆alkyl), —SO₂NH—, —SO₂—N(C₁-C₆alkyl)-, —(C₁-C₆ alkyl)-C(O)NH—, or —(C₁-C₆ alkyl)-C(O)—N(C₁-C₆alkyl)-, wherein the alkyl portion or portions of each of the above is optionally substituted with 1, 2, or 3 groups that are independently halogen, C₁-C₄ alkoxy, amino, mono or di (C₁-C₆ alkyl)amino, OH, indolyl, guanidinyl, carboxyl, or ═O.

In another aspect, the invention provides compounds of formula I-m, i.e., compounds of formula I-1, wherein Y is —NR₂—, —NHC(O)—, —C(O)NH—, —SO₂NH—, or —(C₁-C₆ alkyl)-C(O)—N(C₁-C₆alkyl)-, wherein the alkyl portion or portions of each of the above is optionally substituted with 1, 2, or 3 groups that are independently halogen, C₁-C₄ alkoxy, amino, mono or di (C₁-C₆ alkyl)amino, OH, indolyl, guanidinyl, carboxyl, or ═O.

In another aspect, the invention provides compounds of formula I-n, i.e., compounds of formula I-m, wherein Y is —NR₂—.

In another aspect, the invention provides compounds of formula I-o, i.e., compounds of formula I-m, wherein Y is —NHC(O)—.

In another aspect, the invention provides compounds of formula I-p, i.e., compounds of formula I-m, wherein Y is —C(O)NH—.

In another aspect, the invention provides compounds of formula I-q, i.e., compounds of formula I-m, wherein Y is —SO₂NH—.

In another aspect, the invention provides compounds of formula I-r, i.e., compounds of formula I-m, wherein Y is —(C₁-C₆ alkyl)-C(O)—N(C₁-C₆alkyl)-, wherein the alkyl portion or portions of each of the above is optionally substituted with 1, or 2 groups that are independently halogen, C₁-C₄ alkoxy, amino, mono or di (C₁-C₆ alkyl)amino, OH, indolyl, guanidinyl, carboxyl, or ═O.

In yet another aspect, the invention provides compounds of formula II, i.e., compounds of formula I-e, of the formula:

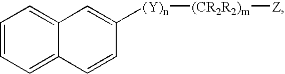

wherein Y, R₂, Z, n and m are as defined herein with regard to compounds of Formula I.

In another aspect, the invention provides compounds of formula II-a, i.e., compounds of formula II, or a pharmaceutically-acceptable salt thereof, wherein
Z is CONR₁OH, and
   R₁ is independently H, or C₁-C₆ alkyl, wherein the alkyl group is optionally substituted with halogen, or C₁-C₆ alkoxy, or phenyl, wherein the phenyl group is optionally substituted with 1, 2, 3, 4, or 5 groups that are independently halogen, C₁-C₆ alkyl, C₁-C₆ alkoxy, phenyl, halogen, nitro, carboxylic acid, C(O)NH₂, C(O)NH(C₁-C₆ alkyl), C(O)N(C₁-C₆ alkyl)(C₁-C₆ alkyl), NH₂, NH(C₁-C₆ alkyl), N(C₁-C₆ alkyl)(C₁-C₆ alkyl), hydroxyl.

In yet another aspect, the invention provides compounds of formula II-b, i.e., compounds of formula II-a, wherein n is 1 and m is 1, 2, or 3.

In yet another aspect, the invention provides compounds of formula II-c, i.e., compounds of formula II-b wherein Z is CONR₁OH, and R₁ is H.

In still yet another aspect, the invention provides compounds of formula II-d, i.e., compounds of formula II-b wherein Z is CONR₁OH, and R₁ is C₁-C₄ alkyl.

In still yet another aspect, the invention provides compounds of formula II-e, i.e., compounds according to any of formulas II, II-a, II-b, II-c, or II-d, wherein m is 1 or 2 and at least one R₂ is hydrogen.

In yet still another aspect, the invention provides a compound of formula II-f, i.e., compounds according to formula II-e, wherein Z is CONR₁OH, R₁ is H, and both R₂ groups are hydrogen.

In still yet another aspect, the invention provides compounds of formula II-g, i.e., compounds of formula II, or a pharmaceutically-acceptable salt thereof, wherein Z is CONHR₁.

In yet still another aspect, the invention provides compounds of formula II-h, i.e., compounds of formula II, II-a, II-b, II-c or II-d, wherein R₂ is independently H, or C₁-C₆ alkyl, where the alkyl group is optionally substituted with one or two groups that are independently halogen, C₁-C₄ alkoxy, phenyl, naphthyl, halogen, nitro, CHO, carboxyl, C(O)NH₂, C(O)NH(C₁-C₆ alkyl), C(O)N(C₁-C₆ alkyl)(C₁-C₆ alkyl), NH₂, NH(C₁-C₆ alkyl), N(C₁-C₆ alkyl)(C₁-C₆ alkyl), hydroxyl, C₁-C₆ alkoxycarbonyl, or nitrile.

In another aspect, the invention provides compounds of formula II, i.e., compounds of formula II-h, wherein one R₂ is H and the other is H or C₁-C₆ alkyl, where the alkyl group is optionally substituted with OH, NH₂, or SH.

In another aspect, the invention provides compounds of formula II-j, i.e., compounds of formula II, II-a, II-b, II-c, II-d, II-e, II-f, II-g, II-h, or II-I, wherein Y is —NR₂—, —(C₁-C₆ alkyl)-NHC(O)—, —(C₁-C₆ alkyl)-N(C₁-C₆ alkyl)C(O)—, —NHC(O)—, —N(C₁-C₆alkyl)C(O)—, —C(O)NH—, —C(O)—N(C₁-C₆alkyl), —SO₂NH—, —SO₂—N(C₁-C₆alkyl)-, —(C₁-C₆ alkyl)-C(O)NH—, or —(C₁-C₆ alkyl)-C(O)—N(C₁-C₆alkyl)-, wherein the alkyl portion or portions of each of the above is optionally substituted with 1, 2, or 3 groups that are independently halogen, C₁-C₄ alkoxy, amino, mono or di (C₁-C₆ alkyl)amino, OH, indolyl, guanidinyl, carboxyl, or ═O.

In another aspect, the invention provides compounds of formula II-k, i.e., compounds of formula II-j, wherein Y is —NR₂—, —NHC(O)—, —C(O)NH—, —SO₂NH—, or —(C₁-C₆ alkyl)-C(O)—N(C₁-C₆alkyl)-, wherein the alkyl portion or portions of each of the above is optionally substituted with 1, 2, or 3 groups that are independently halogen, C₁-C₄ alkoxy, amino, mono or di (C₁-C₆ alkyl)amino, OH, indolyl, guanidinyl, carboxyl, or ═O.

In another aspect, the invention provides compounds of formula II-n, i.e., compounds of formula II-k, wherein Y is —NR₂—.

In another aspect, the invention provides compounds of formula II-o, i.e., compounds of formula II-k, wherein Y is —NHC(O)—.

In another aspect, the invention provides compounds of formula II-p, i.e., compounds of formula II-k, wherein Y is —C(O)NH—.

In another aspect, the invention provides compounds of formula II-q, i.e., compounds of formula II-k, wherein Y is —SO₂NH—.

In another aspect, the invention provides compounds of formula II-r, i.e., compounds of formula II-k, wherein Y is —(C₁-C₆ alkyl)-C(O)—N(C₁-C₆alkyl)-, wherein the alkyl portion or portions of each of the above is optionally substituted with 1, or 2 groups that are independently halogen, C₁-C₄ alkoxy, amino, mono or di (C₁-C₆ alkyl)amino, OH, indolyl, guanidinyl, carboxyl, or ═O.

In yet another aspect, the invention provides compounds of formula III, i.e., compounds of formula I-e, of the formula:

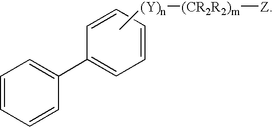

wherein Y, R₂, Z, n and m are as defined herein with regard to compounds of Formula I

In another aspect, the invention provides compounds of formula III-a, i.e., compounds of formula III, or a pharmaceutically-acceptable salt thereof, wherein
Z is CONR₁OH, and
   R₁ is independently H, or C₁-C₆ alkyl, wherein the alkyl group is optionally substituted with halogen, or C₁-C₆ alkoxy, or phenyl, wherein the phenyl group is optionally substituted with 1, 2, 3, 4, or 5 groups that are independently halogen, C₁-C₆ alkyl, C₁-C₆ alkoxy, phenyl, halogen, nitro, carboxylic acid, C(O)NH₂, C(O)NH(C₁-C₆ alkyl), C(O)N(C₁-C₆ alkyl)(C₁-C₆ alkyl), NH₂, NH(C₁-C₆ alkyl), N(C₁-C₆ alkyl)(C₁-C₆ alkyl), hydroxyl.

In yet another aspect, the invention provides compounds of formula II-b, i.e., compounds of formula III-a, wherein n is 1 and m is 1, 2, or 3.

In yet another aspect, the invention provides compounds of formula III-c, i.e., compounds of formula II-b wherein Z is CONR₁OH, and R₁ is H.

In still yet another aspect, the invention provides compounds of formula III-d, i.e., compounds of formula II-b wherein Z is CONR₁OH, and R₁ is C₁-C₄ alkyl.

In still yet another aspect, the invention provides compounds of formula III-e, i.e., compounds according to any of formulas III, III-a, III-b, III-c, or III-d, wherein m is 1 or 2 and at least one R₂ is hydrogen.

In yet still another aspect, the invention provides a compound of formula II-f, i.e., compounds according to formula III-e, wherein Z is CONR₁OH, R₁ is H, and both R₂ groups are hydrogen.

In still yet another aspect, the invention provides compounds of formula III-g, i.e., compounds of formula III, or a pharmaceutically-acceptable salt thereof, wherein Z is CONHR₁.

In yet still another aspect, the invention provides compounds of formula III-h, i.e., compounds of formula III, III-a, III-b, III-c or III-d, wherein R₂ is independently H, or C₁-C₆ alkyl, where the alkyl group is optionally substituted with one or two groups that are independently halogen, C₁-C₄ alkoxy, phenyl, naphthyl, halogen, nitro, CHO, carboxyl, C(O)NH₂, C(O)NH(C₁-C₆ alkyl), C(O)N(C₁-C₆ alkyl)(C₁-C₆ alkyl), NH₂, NH(C₁-C₆ alkyl), N(C₁-C₆ alkyl)(C₁-C₆ alkyl), hydroxyl, C₁-C₆ alkoxycarbonyl, or nitrile.

In another aspect, the invention provides compounds of formula II-i, i.e., compounds of formula II-h, wherein one R₂ is H and the other is H or C₁-C₆ alkyl, where the alkyl group is optionally substituted with OH, NH₂, or SH.

In another aspect, the invention provides compounds of formula III-j, i.e., compounds of formula III, III-a, III-b, III-c, III-d, III-e, III-f, III-g, III-h, or III-I, wherein Y is —NR₂—, —(C₁-C₆ alkyl)-NHC(O)—, —(C₁-C₆ alkyl)-N(C₁-C₆ alkyl)C(O)—, —NHC(O)—, —N(C₁-C₆alkyl)C(O)—, —C(O)NH—, —C(O)—N(C₁-C₆alkyl), —SO₂NH—, —SO₂—N(C₁-C₆alkyl)-, —(C₁-C₆ alkyl)-C(O)NH—, or —(C₁-C₆ alkyl)-C(O)—N(C₁-C₆alkyl)-, wherein the alkyl portion or portions of each of the above is optionally substituted with 1, 2, or 3 groups that are independently halogen, C₁-C₄ alkoxy, amino, mono or di (C₁-C₆ alkyl)amino, OH, indolyl, guanidinyl, carboxyl, or ═O.

In another aspect, the invention provides compounds of formula III-k, i.e., compounds of formula III-j, wherein Y is —NR₂—, —NHC(O)—, —C(O)NH—, —SO₂NH—, or —(C₁-C₆ alkyl)-C(O)—N(C₁-C₆alkyl)-, wherein the alkyl portion or portions of each of the above is optionally substituted with 1, 2, or 3 groups that are independently halogen, C₁-C₄ alkoxy, amino, mono or di (C₁-C₆ alkyl)amino, OH, indolyl, guanidinyl, carboxyl, or ═O.

In another aspect, the invention provides compounds of formula III-n, i.e., compounds of formula III-k, wherein Y is —NR₂—.

In another aspect, the invention provides compounds of formula III-o, i.e., compounds of formula III-k, wherein Y is —NHC(O)—.

In another aspect, the invention provides compounds of formula III-p, i.e., compounds of formula III-k, wherein Y is —C(O)NH—.

In another aspect, the invention provides compounds of formula III-q, i.e., compounds of formula III-k, wherein Y is —SO₂NH—.

In another aspect, the invention provides compounds of formula III-r, i.e., compounds of formula III-k, wherein Y is —(C₁-C₆ alkyl)-C(O)—N(C₁-C₆alkyl)-, wherein the alkyl portion or portions of each of the above is optionally substituted with 1, or 2 groups that are independently halogen, C₁-C₄ alkoxy, amino, mono or di (C₁-C₆ alkyl)amino, OH, indolyl, guanidinyl, carboxyl, or ═O.

In yet another aspect, the invention provides compounds of formula IV, i.e., compounds of formula I-e, of the formula:

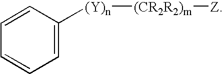

wherein Y, R₂, Z, n and are as defined herein with regard to compounds of Formula I In another aspect, the invention provides compounds of formula IV-a, i.e., compounds of formula IV, or a pharmaceutically-acceptable salt thereof, wherein
Z is CONR₁OH, and
   R₁ is independently H, or C₁-C₆ alkyl, wherein the alkyl group is optionally substituted with halogen, or C₁-C₆ alkoxy, or phenyl, wherein the phenyl group is optionally substituted with 1, 2, 3, 4, or 5 groups that are independently halogen, C₁-C₆ alkyl, C₁-C₆ alkoxy, phenyl, halogen, nitro, carboxylic acid, C(O)NH₂, C(O)NH(C₁-C₆ alkyl), C(O)N(C₁-C₆ alkyl)(C₁-C₆ alkyl), NH₂, NH(C₁-C₆ alkyl), N(C₁-C₆ alkyl)(C₁-C₆ alkyl), hydroxyl.

In yet another aspect, the invention provides compounds of formula IV-b, i.e., compounds of formula IV-a, wherein n is 1 and m is 1, 2, or 3.

In yet another aspect, the invention provides compounds of formula IV-c, i.e., compounds of formula IV-b wherein Z is CONR₁OH, and R₁ is H.

In still yet another aspect, the invention provides compounds of formula IV-d, i.e., compounds of formula IV-b wherein Z is CONR₁OH, and R₁ is C₁-C₄ alkyl.

In still yet another aspect, the invention provides compounds of formula IV-e, i.e., compounds according to any of formulas IV, IV-a, IV-b, IV-c, or IV-d, wherein m is 1 or 2 and at least one R₂ is hydrogen.

In yet still another aspect, the invention provides a compound of formula IV-f, i.e., compounds according to formula IV-e, wherein Z is CONR₁OH, R₁ is H, and both R₂ groups are hydrogen.

In still yet another aspect, the invention provides compounds of formula IV-g, i.e., compounds of formula IV, or a pharmaceutically-acceptable salt thereof, wherein Z is CONHR₁.

In yet still another aspect, the invention provides compounds of formula IV-h, i.e., compounds of formula IV, IV-a, IV-b, IV-c or IV-d, wherein R₂ is independently H, or C₁-C₆ alkyl, where the alkyl group is optionally substituted with one or two groups that are independently halogen, C₁-C₄ alkoxy, phenyl, naphthyl, halogen, nitro, CHO, carboxyl, C(O)NH₂, C(O)NH(C₁-C₆ alkyl), C(O)N(C₁-C₆ alkyl)(C₁-C₆ alkyl), NH₂, NH(C₁-C₆ alkyl), N(C₁-C₆ alkyl)(C₁-C₆ alkyl), hydroxyl, C₁-C₆ alkoxycarbonyl, or nitrile.

In another aspect, the invention provides compounds of formula IV-i, i.e., compounds of formula IV-h, wherein one R₂ is H and the other is H or C₁-C₆ alkyl, where the alkyl group is optionally substituted with OH, NH₂, or SH.

In another aspect, the invention provides compounds of formula IVj, i.e., compounds of formula IV, IV-a, IV-b, IV-c, IV-d, IV-e, IV-f, IV-g, IV-h, or IV-I, wherein Y is —NR₂—, —(C₁-C₆ alkyl)-NHC(O)—, —(C₁-C₆ alkyl)-N(C₁-C₆ alkyl)C(O)—, —NHC(O)—, —N(C₁-C₆alkyl)C(O)—, —C(O)NH—, —C(O)—N(C₁-C₆alkyl), —SO₂NH—, —SO₂—N(C₁-C₆alkyl)-, —(C₁-C₆ alkyl)-C(O)NH—, or —(C₁-C₆ alkyl)-C(O)—N(C₁-C₆alkyl)-, wherein the alkyl portion or portions of each of the above is optionally substituted with 1, 2, or 3 groups that are independently halogen, C₁-C₄ alkoxy, amino, mono or di (C₁-C₆ alkyl)amino, OH, indolyl, guanidinyl, carboxyl, or ═O.

In another aspect, the invention provides compounds of formula IV-k, i.e., compounds of formula IV-j, wherein Y is —NR₂—, —NHC(O)—, —C(O)NH—, —SO₂NH—, or —(C₁-C₆ alkyl)-C(O)—N(C₁-C₆alkyl)-, wherein the alkyl portion or portions of each of the above is optionally substituted with 1, 2, or 3 groups that are independently halogen, C₁-C₄ alkoxy, amino, mono or di (C₁-C₆ alkyl)amino, OH, indolyl, guanidinyl, carboxyl, or ═O.

In another aspect, the invention provides compounds of formula IV-n, i.e., compounds of formula IV-k, wherein Y is —NR₂—.

In another aspect, the invention provides compounds of formula IV-o, i.e., compounds of formula IV-k, wherein Y is —NHC(O)—.

In another aspect, the invention provides compounds of formula IV-p, i.e., compounds of formula IV-k, wherein Y is —C(O)NH—.

In another aspect, the invention provides compounds of formula IV-q, i.e., compounds of formula IV-k, wherein Y is —SO₂NH—.

In another aspect, the invention provides compounds of formula IV-r, i.e., compounds of formula IV-k, wherein Y is —(C₁-C₆ alkyl)-C(O)—N(C₁-C₆alkyl)-, wherein the alkyl portion or portions of each of the above is optionally substituted with 1, or 2 groups that are independently halogen, C₁-C₄ alkoxy, amino, mono or di (C₁-C₆ alkyl)amino, OH, indolyl, guanidinyl, carboxyl, or ═O.

In certain other aspect, the invention provides compounds of formula B, wherein

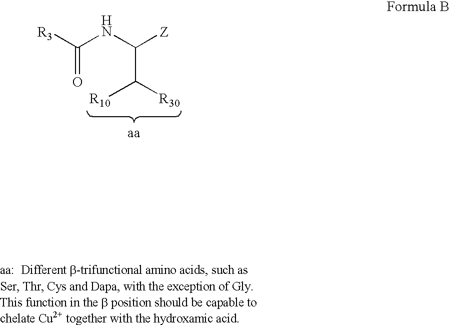

wherein n, R₃, and Z are as defined in Formula I;
R₁₀ is H, alkyl optionally substituted with OH, SH, amino, NH(C₁-C₆ alkyl), or N(C₁-C₆ alkyl)(C₁-C₆ alkyl); and
R₃₀ is H or C₁-C₄ alkyl (in one aspect, R₃₀ is H; in another aspect, R₃₀ is methyl).

In another aspect, the invention provides compounds of formula B-1, i.e., compounds of formula B wherein R₃ is C₁-C₄ alkyl substituted with phenyl, where the phenyl is optionally substituted with 1, 2, or 3 groups that are independently C₁-C₆ alkyl (in one aspect, C₁-C₄ alkyl, in another aspect, C₁-C₂ alkyl), OH, C₁-C₆ alkoxy, or phenyl; or
R₃ is C₃-C₆ cycloalkyl optionally substituted with phenyl, where the phenyl is optionally substituted with 1, 2, or 3 groups that are independently C₁-C₆ alkyl (in one aspect, C₁-C₄ alkyl, in another aspect, C₁-C₂ alkyl), OH, C₁-C₆ alkoxy, or phenyl.

In yet another aspect, the invention provides compounds of formula B-2, i.e., compounds of formula B wherein R₃ is C₂-C₃ alkenyl substituted with furanyl or phenyl, where the phenyl is optionally substituted with 1, 2, or 3 groups that are independently C₁-C₆ alkyl (in one aspect, C₁-C₄ alkyl, in another aspect, C₁-C₂ alkyl), OH, C₁-C₆ alkoxy, or phenyl.

In still yet another aspect, the invention provides compounds of formula B-3, i.e., compounds of formula B wherein R₃ is indolyl, or phenyl, where the phenyl is optionally substituted with 1, 2, or 3 groups that are independently C₁-C₆ alkyl (in one aspect, C₁-C₄ alkyl, in another aspect, C₁-C₂ alkyl), OH, C₁-C₆ alkoxy, or phenyl.

In yet still another aspect, the invention provides compounds of formula B-4, i.e., compounds of formula B, B-1, B-2, or B-3, wherein Z is —C(O)NHOH.

In certain other aspect, the invention provides compounds of formula C, wherein

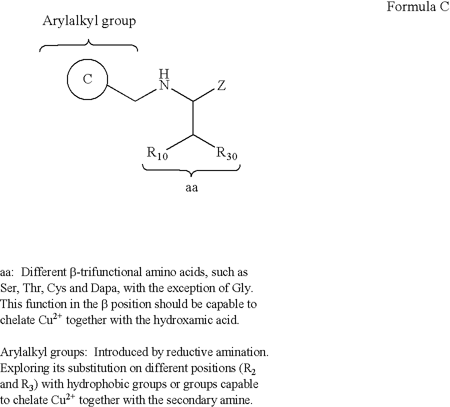

wherein Z is as defined in formula I;
R₁₀ is H, amino, mono or di (C₁-C₆ alkyl)amino, OH, or SH;
R₃₀ is H or C₁-C₄ alkyl (in one aspect, methyl); and
the C-ring is phenyl or naphthyl, each of which is optionally substituted with one or more groups that are independently OH, NO₂, halogen (in one aspect, F), C₁-C₆ alkyl (in one aspect, methyl), C₁-C₆ alkoxy (in one aspect, methoxy), amino, mono or di (C₁-C₆ alkyl)amino, phenyl, phenyl C₁-C₄ alkoxy (in one aspect, benzyloxy).

In another aspect, the invention provides compounds of formula C-1, i.e., compounds of formula C, wherein the C-ring is phenyl optionally substituted with one or more groups that are independently OH, NO₂, halogen (in one aspect, F), C₁-C₆ alkyl (in one aspect, methyl), C₁-C₆ alkoxy (in one aspect, methoxy), amino, mono or di (C₁-C₆ alkyl)amino, phenyl, phenyl C₁-C₄ alkoxy (in one aspect, benzyloxy).

In another aspect, the invention provides compounds of formula C-2, i.e., compounds of formula C, wherein the C-ring is naphthyl optionally substituted with one or more groups that are independently OH, NO₂, halogen (in one aspect, F), C₁-C₆ alkyl (in one aspect, methyl), C₁-C₆ alkoxy (in one aspect, methoxy), amino, mono or di (C₁-C₆ alkyl)amino, phenyl, phenyl C₁-C₄ alkoxy (in one aspect, benzyloxy).

In another aspect, the invention provides compounds of formula C-3, i.e., compounds of formula C, wherein Z is C(O)NHOH.

In certain other aspect, the invention provides compounds of formula D, wherein

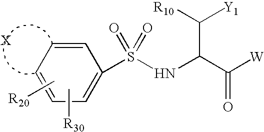

each R₁₀ is independently H, C₁-C₆ alkyl, C₂-C₆ alkenyl, C₂-C₆ alkynyl, aryl, substituted aryl, heterocycloalkyl containing at least one and no more than two heteroatoms selected from S, N, and O, or C₃-C₇ cycloalkyl, and where any member of the alkyl, aryl/or cycloalkyl group is optionally substituted with halogen, C₁-C₆ alkyl or C₁-C₆ alkoxy, aryl or substituted aryl;
R₂₀ and R₃₀ are independently H, C₁-C₆ alkyl, aryl, substituted aryl, C₁-C₆ alkoxy, C₁-C₆ alkoxyalkyl, C₃-C₇ cycloalkyl, C₃-C₇ cycloalkylalkyl, C₃-C₇ cycloalkylalkoxy, heteroaryl, heteroarylalkyl, heterocycloalkyl, or heterocycloalkylalkyl, where each of the above is optionally substituted with 1, 2, 3, 4, or 5 groups that are independently halogen, C₁-C₆ alkyl, C₁-C₆ alkoxy, aryl, substituted aryl, halogen, nitro, nitroso, aldehyde, carboxylic acid, —C(O)NH₂, —C(O)NH(C₁-C₆ alkyl), —C(O)N(C₁-C₆ alkyl)(C₁-C₆ alkyl), amino, mono or di (C₁-C₆ alkyl)amino, C₁-C₆ alkoxycarbonyl, sulfate, imine, hydroxyl or nitrile; and
W is NHOH, NH₂, NHR₁₀, OR₁₀, NH—NHR₁₀;
X is C, CH, or any heteroatom selected from S, N, and O;
Y₁ is OR₁₀, NHR₁₀, or SR₁₀;
   the dashed lines represent a fused aryl or heterocycloalkyl ring that are optionally present.

In another aspect, the invention provides compounds of formula D-1, i.e., compounds of formula D wherein R₁₀ is H, C₁-C₆ alkyl.

In another aspect, the invention provides compounds of formula D-2, i.e., compounds of formula D-1 wherein Y, is OH, NH₂, or SH.

In another aspect, the invention provides compounds of formula D-3, i.e., compounds of formula D-2 wherein R₂₀ and R₃₀ are independently H, C₁-C₆ alkyl, phenyl, C₁-C₆ alkoxy, NO₂, CF₃, amino, mono or di (C₁-C₄ alkyl)amino.

In another aspect, the invention provides compounds of formula D-4, i.e., compounds of formula D-3 wherein the

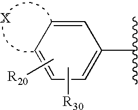

portion of the molecule is a naphthyl group optionally substituted with R₂₀ and R₃₀.

In another aspect, the invention provides compounds of formula D-5, i.e., compounds of formula D-3 wherein the

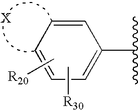

portion of the molecule is a quinolinyl group optionally substituted with R₂₀ and R₃₀.

In another aspect, the invention provides compounds of formula D-6, i.e., compounds of formula D, D-1, D-2, D-3, D-4, or D-5, wherein W is NHOH.

In certain other aspect, the invention provides compounds of formula F, wherein

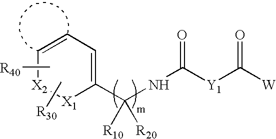

R₁₀ and R₂₀ are independently H, C₁-C₆ alkyl, C₂-C₆ alkenyl, C₂-C₆ alkynyl, aryl, substituted aryl, heterocycloalkyl containing at least one and no more than two heteroatoms selected from S, N, and O, or C₃-C₇ cycloalkyl, and where any member of the alkyl, aryl/or cycloalkyl group is optionally substituted with halogen, C₁-C₆ alkyl or C₁-C₆ alkoxy, aryl or substituted aryl;
R₃₀ and R₄₀ are independently H, OH, SH, halogen, nitro, amino, mono or di(C₁-C₆ alkyl)amino, C₁-C₆ alkyl, C₁-C₆ alkanoyl, aryl, substituted aryl, C₁-C₆ alkoxy, C₁-C₆ alkoxyalkyl, C₃-C₇ cycloalkyl, C₃-C₇ cycloalkylalkyl, C₃-C₇ cycloalkylalkoxy, heteroaryl, heteroarylalkyl, heterocycloalkyl, or heterocycloalkylalkyl, where the cyclic portion, the alkyl portion or a combination thereof of each of the above is optionally substituted with 1, 2, 3, 4, or 5 groups that are independently halogen, C₁-C₆ alkyl, C₁-C₆ alkoxy, aryl, substituted aryl, halogen, nitro, nitroso, aldehyde, carboxylic acid, —C(O)NH₂, —C(O)NH(C₁-C₆ alkyl), —C(O)N(C₁-C₆ alkyl)(C₁-C₆ alkyl), amino, mono or di(C₁-C₆ alkyl)amino, C₁-C₆ alkoxycarbonyl, sulfate, imine, hydroxyl or nitrile;
Y₁ is (CH₂)ₙ or aryl;
m is 0, 1, 2, 3, or 4;
n is 0, 1, 2, 3, or 4;
W is NHOH, NH₂, NHR₁₀, OR₁₀, or NH—NHR₁₀;
X₁ and X₂ are independently C, CH, or N, provided that X₁ and X₂ are not simultaneously N; where the dashed lines represent a cycloalkyl or aryl group that is optionally present; and n and m independently an integer from 1 to 5.

In another aspect, the invention provides compounds of formula F-1, i.e., compounds of formula F, wherein m is 0.

In yet another aspect, the invention provides compounds of formula F-2, i.e., compounds of formula F-1, wherein R₃₀ and R₄₀ are independently H, OH, SH, halogen, nitro, amino, mono or di(C₁-C₆ alkyl)amino, C₁-C₆ alkyl, C₁-C₆ alkanoyl, or C₁-C₆ alkoxy.

In still another aspect, the invention provides compounds of formula F-3, i.e., compounds of formula F-2, wherein Y₁ is (CH₂)ₙ, and n is 0, 1, or 2 (in one aspect, 2).

In yet still another aspect, the invention provides compounds of formula F-4, i.e., compounds of formula F-2, wherein Y₁ is phenyl, naphthyl, or biphenyl.

In yet still another aspect, the invention provides compounds of formula F-5, i.e., compounds of formula F-3, or F-4, wherein W is NHOH.

In certain other aspect, the invention provides compounds of formula I, wherein

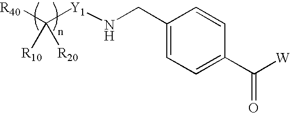

R₁₀ is independently H, C₁-C₆ alkyl, aryl, C₂-C₆ alkenyl, C₂-C₆alkynyl, substituted aryl, heterocycloalkyl containing at least one and no more than two heteroatoms selected from S, N, and O, or C₃-C₇ cycloalkyl, and where any member of the alkyl, aryl/or cycloalkyl group is optionally substituted with halogen, C₁-C₆ alkyl or C₁-C₆ alkoxy, aryl or substituted aryl;
R₂₀ and R₄₀ are independently H, C₁-C₆ alkyl, aryl, substituted aryl, C₁-C₆ alkoxy, C₁-C₆ alkoxyalkyl, C₃-C₇ cycloalkyl, C₃-C₇ cycloalkylalkyl, C₃-C₇ cycloalkylalkoxy, heteroaryl, heteroarylalkyl, heterocycloalkyl, or heterocycloalkylalkyl, where each of the above is optionally substituted with 1, 2, 3, 4, or 5 groups that are independently halogen, C₁-C₆ alkyl, C₁-C₆ alkoxy, aryl, arylalkoxy (in one aspect, benzyloxy), substituted aryl, halogen, nitro, nitroso, aldehyde, carboxylic acid, —C(O)NH₂, —C(O)NH(C₁-C₆ alkyl), —C(O)N(C₁-C₆ alkyl)(C₁-C₆ alkyl), amino, C₁-C₆ alkoxycarbonyl, sulfate, imine, hydroxyl or nitrile; and
W is NHOH, NH₂O, NHR₁₀, OR₁₀, NH—NHR₁₀;
X is any heteroatom selected from S, N, and O; or
R₁₀, R₂₀ and the carbon to which they are attached form a cycloalkyl ring (preferably C₅-C₆ cycloalkyl);
Y₁ is SO₂, C(O), CH₂, —NHC(O), and
n an integer from 1 to 5.

In another aspect, the invention provides compounds of formula I-1, i.e., compounds of formula I, wherein R₄₀ is phenyl, naphthyl, furanyl, indolyl, or quinolinyl, where each of the above is optionally substituted with 1, 2, or 3 groups that are independently halogen, C₁-C₆ alkyl, C₁-C₆ alkoxy, phenyl, halogen, nitro, carboxylic acid, —C(O)NH₂, —C(O)NH(C₁-C₆ alkyl), —C(O)N(C₁-C₆ alkyl)(C₁-C₆ alkyl), amino, C₁-C₆ alkoxycarbonyl, hydroxyl or nitrile.

In yet still another aspect, the invention provides compounds of formula I-2, i.e., compounds of formula I-1, wherein n is 1 or 2 and R₁₀ and R₂₀ are both H.

In still yet another aspect, the invention provides compounds of formula I-3, i.e., compounds of formula I-1, wherein n is 0.

In another aspect, the invention provides compounds of formula I-4, i.e., compounds of formula I-1 or 1-2, wherein W is NHOH.

In still yet another aspect, the invention provides compounds of formula I-5, i.e., compounds of formula I-1, wherein R₁₀, R₂₀ and the carbon to which they are attached form a cycloalkyl ring (preferably C₅-C₆ cycloalkyl) and W is NHOH.

In certain other aspect, the invention provides compounds of formula J, wherein

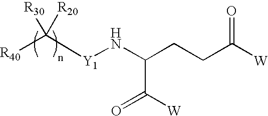

R₁₀ is independently H, C₁-C₆ alkyl, aryl, C₂-C₆ alkenyl, C₂-C₆alkynyl, substituted aryl, heterocycloalkyl containing at least one and no more than two heteroatoms selected from S, N, and O, or C₃-C₇ cycloalkyl, and where any member of the alkyl, aryl/or cycloalkyl group is optionally substituted with halogen, C₁-C₆ alkyl or C₁-C₆ alkoxy, aryl or substituted aryl;
R₂₀ is independently H, C₁-C₆ alkyl, C₂-C₆ alkenyl, C₂-C₆ alkynyl, aryl, substituted aryl, heterocycloalkyl containing at least one and no more than two heteroatoms selected from S, N, and O, or C₃-C₇ cycloalkyl, and where any member of the alkyl, aryl/or cycloalkyl group is optionally substituted with halogen, C₁-C₆ alkyl or C₁-C₆ alkoxy, aryl or substituted aryl;
R₃₀ and R₄₀ are independently H, C₁-C₆ alkyl, aryl (in one aspect, phenyl), substituted aryl, C₁-C₆ alkoxy, C₁-C₆ alkoxyalkyl, C₃-C₇ cycloalkyl, C₃-C₇ cycloalkylalkyl, C₃-C₇ cycloalkylalkoxy, heteroaryl, heteroarylalkyl, heterocycloalkyl, or heterocycloalkylalkyl, where each of the above is optionally substituted with 1, 2, 3, 4, or 5 groups that are independently halogen, C₁-C₆ alkyl, C₁-C₆ alkoxy, aryl, substituted aryl, halogen, nitro, nitroso, aldehyde, carboxylic acid, —C(O)NH₂, —C(O)NH(C₁-C₆ alkyl), —C(O)N(C₁-C₆ alkyl)(C₁-C₆ alkyl), amino, C₁-C₆ alkoxycarbonyl, sulfate, imine, hydroxyl or nitrile;
W at each occurrence is independently NHOH, NHR₁₀, OR₁₀, or NH—NHR₁₀;
Y, is CH₂, C(O), or SO₂;

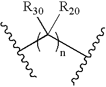

also encompasses olefins when n is at least 2, in such a case, R₂₀ is not present on the olefinic carbons; or
R₂₀, R₃₀, and the carbon to which they are attached form a cycloalkyl ring (in one aspect, a C₅-C₆ cycloalkyl ring) and
n is an integer from 1 to 5.

In another aspect, the invention provides compounds of formula J-1, i.e., compounds of formula J, wherein Y₁ is C(O).

In yet another aspect, the invention provides compounds of formula J-2, i.e., compounds of formula J-1, wherein at least one W is OR₁₀ (in one aspect, R₁₀ is H or C₁-C₆ alkyl; in another aspect, both W groups are OH).

In still another aspect, the invention provides compounds of formula J-3, i.e., compounds of formula J-2, wherein R₄₀ is phenyl, indolyl, or furanyl, where each of the above is optionally substituted with 1, 2, or 3 groups that are independently halogen, C₁-C₆ alkyl (in one aspect, C₁-C₂ alkyl), C₁-C₆ alkoxy (in one aspect, C₁-C₂ alkoxy), aryl (in one aspect, phenyl), halogen, nitro, carboxylic acid, —C(O)NH₂, —C(O)NH(C₁-C₆ alkyl), —C(O)N(C₁-C₆ alkyl)(C₁-C₆ alkyl), amino, C₁-C₆ alkoxycarbonyl, hydroxyl or nitrile.

In yet still another aspect, the invention provides compounds of formula J-4, i.e., compounds of formula J-3, wherein when n is 2;

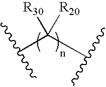

is a C₂ olefin; R₂₀ is absent and R₃₀ is H or C₁-C₄ alkyl.

In still another aspect, the invention provides compounds of formula J-5, i.e., compounds of formula J-3, wherein R₂₀, R₃₀, and the carbon to which they are attached form a cycloalkyl ring (in one aspect, a C₅ cycloalkyl ring) and at least one W is OH (in another aspect, both W groups are OH).

In certain other aspect, the invention provides compounds of formula K, wherein

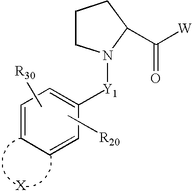

R₂₀ and R₃₀ are independently H, C₁-C₆ alkyl, aryl, substituted aryl, heterocycloalkyl containing at least one and no more than two heteroatoms selected from S, N, and O, or C₃-C₇ cycloalkyl, and where any member of the alkyl, aryl/or cycloalkyl group is optionally substituted with halogen, OH, SH, C₁-C₆ alkyl or C₁-C₆ alkoxy, aryl (in one aspect, phenyl) or substituted aryl;
W is NHOH, NHR₁₀, OR₁₀, or NH—NHR₁₀;
X is C, CH, or any heteroatom selected from S, N, and O;
Y₁ is CO, or CH₂; and
   the dashed lines represent a fused heteroaryl or heterocycloalkyl ring, which is optionally present.

In another aspect, the invention provides compounds of formula K-1, i.e., compounds of formula K, wherein Y₁ is CO.

In still another aspect, the invention provides compounds of formula K-2, i.e., compounds of formula K-1, wherein

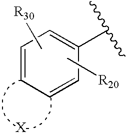

is phenyl, naphthyl, pyridyl, or quinolinyl, each of which is optionally substituted with 1, 2, or 3 groups that are independently OH, SH, C₁-C₄ alkyl, C₁-C₄ alkoxy, or phenyl.

In yet still another aspect, the invention provides compounds of formula K-2, i.e., compounds of formula K-1, wherein W is NHOH or NH₂.

In certain other aspect, the invention provides compounds of formula M, wherein

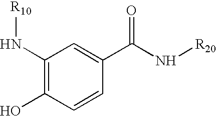

R₁₀ and R₂₀ are independently H, C₁-C₆ alkyl, C₂-C₆ alkenyl, C₂-C₆ alkynyl, aryl (in one aspect, phenyl), substituted aryl, heterocycloalkyl containing at least one and no more than two heteroatoms selected from S, N, and O, or C₃-C₇ cycloalkyl, and where any member of the alkyl, aryl/or cycloalkyl group is optionally substituted with halogen, C₁-C₆ alkyl or C₁-C₆ alkoxy, aryl or substituted aryl.

In another aspect, the invention provides compounds of formula N-1, i.e., compounds of formula M, wherein R₁₀ is H or C₁-C₄ alkyl.

In still another aspect, the invention provides compounds of formula N-2, i.e., compounds of formula N-1, wherein R₂₀ is C₁-C₆ alkyl substituted with phenyl or naphthyl, where the each is optionally substituted with 1 or 2 groups that are independently OH, halogen, C₁-C₄ alkyl (in one aspect, methyl), or C₁-C₄ alkoxy (in one aspect, methoxy).

In certain other aspect, the invention provides compounds of formula N, wherein

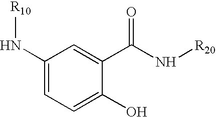

R₁₀ and R₂₀ are independently H, C₁-C₆ alkyl, C₂-C₆ alkenyl, C₂-C₆ alkynyl, aryl (in one aspect, phenyl), substituted aryl, heterocycloalkyl containing at least one and no more than two heteroatoms selected from S, N, and O, or C₃-C₇ cycloalkyl, and where any member of the alkyl, aryl/or cycloalkyl group is optionally substituted with halogen, C₁-C₆ alkyl or C₁-C₆ alkoxy, aryl or substituted aryl.

In another aspect, the invention provides compounds of formula N-1, i.e., compounds of formula N, wherein R₁₀ is H or C₁-C₄ alkyl.

In still another aspect, the invention provides compounds of formula N2, i.e., compounds of formula N-1, wherein R₂₀ is C₁-C₆ alkyl substituted with phenyl or naphthyl, where the each is optionally substituted with 1 or 2 groups that are independently OH, halogen, C₁-C₄ alkyl (in one aspect, methyl), or C₁-C₄ alkoxy (in one aspect, methoxy).

### DEFINITIONS

By “alkyl”, and “C₁-C₆ alkyl” in the present invention is meant straight or branched chain alkyl groups having 1-6 carbon atoms, such as, methyl, ethyl, propyl, isopropyl, n-butyl, sec-butyl, tert-butyl, pentyl, 2-pentyl, isopentyl, neopentyl, hexyl, 2-hexyl, 3-hexyl, and 3-methylpentyl.

By “alkoxy”, and “C₁-C₆ alkoxy” in the present invention is meant straight or branched chain alkoxy groups having 1-6 carbon atoms, such as, for example, methoxy, ethoxy, propoxy, isopropoxy, n-butoxy, sec-butoxy, tert-butoxy, pentoxy, 2-pentyl, isopentoxy, neopentoxy, hexoxy, 2-hexoxy, 3-hexoxy, and 3-methylpentoxy.

By the term “halogen” in the present invention is meant fluorine, bromine, chlorine, and iodine.

By “cycloalkyl”, e.g., C₃-C₇ cycloalkyl, in the present invention is meant cycloalkyl groups having 3-7 atoms such as, for example cyclopropyl, cyclobutyl, cyclopentyl, cyclohexyl, and cycloheptyl.

By “aryl” is meant an aromatic carbocyclic group having a single ring (e.g., phenyl), multiple rings (e.g., biphenyl, naphthyl, anthryl, or phenanthryl), or multiple condensed rings in which at least one is aromatic, (e.g., 1,2,3,4-tetrahydronaphthyl), where each aryl group is optionally mono-, di-, or trisubstituted with groups that are independently, e.g., halogen, NO₂, amino, NH(C₁-C₆ alkyl), N(C₁-C₆ alkyl)(C₁-C₆ alkyl), C₁-C₆ alkyl, C₁-C₆ alkoxy, C₁-C₆ alkylthio, trifluoromethyl, C₁-C₆ acyloxy, aryl (in one aspect, phenyl), heteroaryl (in one aspect, pyridyl, indolyl, or furanyl), and hydroxy. Preferred aryl groups include phenyl, biphenyl, and naphthyl, each of which is optionally substituted as defined herein. More preferred aryl groups include phenyl and naphthyl, each of which is optionally substituted as defined herein.

By “heteroaryl” is meant an aromatic ring or aromatic ring system, wherein each ring contains of 5-, 6-, or 7-members wherein at least one and up to four ring members are selected from nitrogen, oxygen, or sulfur, and where the heteroaryl group is optionally mono, di, or trisubstituted with groups that are independently, e.g., halogen, NO₂, amino, NH(C₁-C₆ alkyl), N(C₁-C₆ alkyl)(C₁-C₆ alkyl), C₁-C₆ alkyl, C₁-C₆ alkoxyl C₁-C₆ alkylthio, trifluoromethyl, C₁-C₆ acyloxy, aryl (in one aspect, phenyl), heteroaryl (in one aspect, pyridyl, indolyl, or furanyl), and hydroxy. Such heteroaryl groups include, for example, thienyl, furanyl, thiazolyl, imidazolyl, (is)oxazolyl, pyrrolyl, pyrazolyl, imidazolyl, pyridyl, pyrimidinyl, (iso)quinolinyl, indolyl, napthyridinyl, benzimidazolyl, and benzoxazolyl. Preferred heteroaryls are thiazolyl, pyrimidinyl, pyrimidin-2-yl, indolyl, pyridyl, 1-imidazolyl, 2-thienyl, 1-, or 2-quinolinyl, 1-, or 2-isoquinolinyl, 1-, or 2-tetrahydro isoquinolinyl, 2- or 3-furanyl, imidazolyl, and 2-tetrahydrofuranyl.

By “heterocycloalkyl,” is meant one or more carbocyclic ring systems of 3, 4, 5, 6, or 7-membered rings which includes fused ring systems of 9-11 atoms containing at least one and up to four heteroatoms selected from nitrogen, oxygen, and sulfur, and where each heterocycloalkyl group is where each aryl group is optionally mono-, di-, or trisubstituted with groups that are independently, e.g., halogen, NO₂, amino, NH(C₁-C₆ alkyl), N(C₁-C₆ alkyl)(C₁-C₆ alkyl), C₁-C₆ alkyl, C₁-C₆ alkoxy, C₁-C₆ alkylthio, trifluoromethyl, C₁-C₆ acyloxy, aryl (in one aspect, phenyl), heteroaryl (in one aspect, pyridyl, indolyl, or furanyl), and hydroxy. Preferred heterocycles of the present invention include morpholinyl, thiomorpholinyl, thiomorpholinyl S-oxide, thiomorpholinyl S,S-dioxide, piperazinyl, homopiperazinyl, pyrrolidinyl, pyrrolinyl, tetrahydropyranyl, piperidinyl, tetrahydrofuranyl, tetrahydrothienyl, homopiperidinyl, homomorpholinyl, homothiomorpholinyl, homothiomorpholinyl S,S-dioxide, oxazolidinonyl, dihydropyrazolyl, dihydropyrrolyl, dihydropyrazinyl, dihydropyridinyl, dihydropyrimidinyl, dihydrofuryl, dihydropyranyl, azepanyl, diazepanyl, tetrahydrothienyl S-oxide, tetrahydrothienyl S,S-dioxide and homothiomorpholinyl S-oxide.

Compounds of formula I are useful as pharmaceutical agents, and can be provided as pharmaceutical compositions. The pharmaceutical compositions can be manufactured in a manner that is itself known, e.g., by means of a conventional mixing, dissolving, granulating, dragee-making, levigating, emulsifying, encapsulating, entrapping or lyophilizing processes.

Pharmaceutical compositions can be formulated in conventional manner using one or more physiologically acceptable carriers comprising excipients and auxiliaries that facilitate processing of the active compounds into preparations that can be used pharmaceutically. Proper formulation is dependent upon the route of administration chosen.

Non-toxic pharmaceutical salts include salts of acids such as hydrochloric, phosphoric, hydrobromic, sulfuric, sulfinic, formic, toluenesulfonic, methanesulfonic, nitic, benzoic, citric, tartaric, maleic, hydroiodic-, alkanoic such as acetic, HOOC—(CH₂)ₙ—CH₃ where n is 0-4, and the like. Non-toxic pharmaceutical base addition salts include salts of bases such as sodium, potassium, calcium, ammonium, and the like. Those skilled in the art will recognize a wide variety of non-toxic pharmaceutically acceptable addition salts.

For injection, the compounds prepared according to the methods of the invention can be formulated in appropriate aqueous solutions, such as physiologically compatible buffers such as Hanks's solution, Ringer's solution, or physiological saline buffer. For transmucosal and transcutaneous administration, penetrants appropriate to the barrier to be permeated are used in the formulation. Such penetrants are generally known in the art.

For oral administration, the compounds can be formulated readily by combining the active compounds with pharmaceutically acceptable carriers well-known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethylcellulose, sodium carboxymethylcellulose, and/or polyvinylpyrrolidone (PVP). If desired, disintegrating agents can be added, such as the cross-linked polyvinyl pyrrolidone, agar, or alginic acid or a salt thereof such as sodium alginate.

Dragee cores are provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration. For buccal administration, the compositions can take the form of tablets or lozenges formulated in conventional manner.

For administration by inhalation, the compounds prepared according to the methods of the invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g., gelatin for use in an inhaler or insufflator can be formulated containing a powder mix of the compound and a suitable powder base such as lactose or starch.

The compounds can be formulated for parenteral administration by injection, e.g., by bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

Pharmaceutical formulations for parenteral administration include aqueous solutions of the active compounds in water-soluble form. Additionally, suspensions of the active compounds can be prepared as appropriate oily injection suspensions. Suitable lipophilic solvents or vehicles include fatty oils such as sesame oil, or synthetic fatty acid esters, such as ethyloleate or triglycerides, or liposomes. Aqueous injection suspensions can contain substances that increase the viscosity of the suspension, such as sodium carboxymethyl cellulose, sorbitol, or dextran. Optionally, the suspension can also contain suitable stabilizers or agents that increase the solubility of the compounds to allow for the preparation of highly concentrated solutions. Alternatively, the active ingredient can be in powder form for constitution with a suitable vehicle, e.g., sterile pyrogen-free water, before use. The compounds can also be formulated in rectal compositions such as suppositories or retention enemas, e.g., containing conventional suppository bases such as cocoa butter or other glycerides.

In addition to the formulations described previously, the compounds can also be formulated as a depot preparation. Such long acting formulations can be administered by implantation (for example subcutaneously or intramuscularly) or by intramuscular injection. Thus, for example, the compounds can be formulated with suitable polymeric or hydrophobic materials (for example as an emulsion in acceptable oil) or ion exchange resins, or as sparingly soluble derivatives, for example, as a sparingly soluble salt.

A pharmaceutical carrier for hydrophobic compounds of formula I is a cosolvent system comprising benzyl alcohol, a nonpolar surfactant, a water-miscible organic polymer, and an aqueous phase. The cosolvent system can be the VPD co-solvent system. VPD is a solution of 3% w/v benzyl alcohol, 8% w/v of the nonpolar surfactant polysorbate 80, and 65% w/v polyethylene glycoL300, made up to volume in absolute ethanol. The VPD co-solvent system (VPD:5W) consists of VPD diluted 1:1 with a 5% dextrose in water solution. This co-solvent system dissolves hydrophobic compounds well, and itself produces low toxicity upon systemic administration. Naturally, the proportions of a co-solvent system can be varied considerably without destroying its solubility and toxicity characteristics. Furthermore, the identity of the co-solvent components can be varied: for example, other low-toxicity nonpolar surfactants can be used instead of polysorbate 80; the fraction size of polyethylene glycol can be varied; other biocompatible polymers can replace polyethylene glycol, e.g. polyvinyl pyrrolidone; and other sugars or polysaccharides can substitute for dextrose.

Alternatively, other delivery systems for hydrophobic pharmaceutical compounds can be employed. Liposomes and emulsions are well known examples of delivery vehicles or carriers for hydrophobic drugs. Certain organic solvents such as dimethylsulfoxide also can be employed, although usually at the cost of greater toxicity. Additionally, the compounds can be delivered using a sustained-release system, such as semipermeable matrices of solid hydrophobic polymers containing the therapeutic agent. Various sustained-release materials have been established and are well known by those skilled in the art. Sustained-release capsules can, depending on their chemical nature, release the compounds for a few weeks up to over 100 days. Depending on the chemical nature and the biological stability of the therapeutic reagent, additional strategies for protein and nucleic acid stabilization can be employed.

The pharmaceutical compositions also can comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

The compounds of Formula I can be provided as salts with pharmaceutically compatible counterions. Pharmaceutically compatible salts can be formed with many acids, including but not limited to hydrochloric, sulfuric, acetic, lactic, tartaric, malic, succinic, phosphoric, hydrobromic, sulfinic, formic, toluenesulfonic, methanesulfonic, nitic, benzoic, citric, tartaric, maleic, hydroiodic, alkanoic such as acetic, HOOC—(CH₂)ₙ—CH₃ where n is 0-4, and the like. Salts tend to be more soluble in aqueous or other protonic solvents that are the corresponding free base forms. Non-toxic pharmaceutical base addition salts include salts of bases such as sodium, potassium, calcium, ammonium, and the like. Those skilled in the art will recognize a wide variety of non-toxic pharmaceutically acceptable addition salts.

Pharmaceutical compositions of the compounds prepared according to the methods of the invention can be formulated and administered through a variety of means, including systemic, localized, or topical administration. Techniques for formulation and administration can be found in “Remington's Pharmaceutical Sciences,” Mack Publishing Co., Easton, Pa. The mode of administration can be selected to maximize delivery to a desired target site in the body. Suitable routes of administration can, for example, include oral, rectal, transmucosal, transcutaneous, or intestinal administration; potential delivery, including intramuscular, subcutaneous, intramedullary injections, as well as intrathecal, direct intraventricular, intravenous, intraperitoneal, intranasal, or intraocular injections.

Alternatively, one can administer the compound in a local rather than systemic manner, for example, via injection of the compound directly into a specific tissue, often in a depot or sustained release formulation.

Pharmaceutical compositions suitable for use include compositions wherein the active ingredients are contained in an effective amount to achieve its intended purpose. More specifically, a therapeutically effective amount means an amount effective to prevent development of or to alleviate the existing symptoms of the subject being treated. Determination of the effective amounts is well within the capability of those skilled in the art, especially in light of the detailed disclosure provided herein.

For administration to non-human animals, the drug or a pharmaceutical composition containing the drug may also be added to the animal feed or drinking water. It will be convenient to formulate animal feed and drinking water products with a predetermined dose of the drug so that the animal takes in an appropriate quantity of the drug along with its diet. It will also be convenient to add a premix containing the drug to the feed or drinking water approximately immediately prior to consumption by the animal.

Preferred compounds prepared according to the methods of the invention will have certain pharmacological properties. Such properties include, but are not limited to oral bioavailability, low toxicity, low serum protein binding and desirable in vitro and in vivo half-lives. Assays may be used to predict these desirable pharmacological properties. Assays used to predict bioavailability include transport across human intestinal cell monolayers, including Caco-2 cell monolayers. Serum protein binding may be predicted from albumin binding assays. Such assays are described in a review by Oraveova et al. (1996, Journal of Chromatography B-Biomedical Applications 677:1-28). Compound half-life is inversely proportional to the frequency of dosage of a compound. In vitro half-lives of compounds may be predicted from assays of microsomal half-life as described by Kuhnz and Gieschen (1998, Drug Metabolism and Disposition 26:1120-1127).

Toxicity and therapeutic efficacy of such compounds can be determined by conventional pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio between LD₅₀ and ED₅₀. Compounds that exhibit high therapeutic indices are preferred. The data obtained from these cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. The exact formulation, route of administration and dosage can be chosen by the individual physician in view of the patient's condition. (See, e.g. Fing et al, 1975, in THE PHARMACOLOGICAL BASIS OF THERAPEUTICS, Ch. 1, p. 1).

Dosage amount and interval can be adjusted individually to provide plasma levels of the active moiety that are sufficient to maintain bacterial cell growth-inhibitory effects. Usual patient dosages for systemic administration range from 100-2000 mg/day. Stated in terms of patient body surface areas, usual dosages range from 50-910 mg/m²/day. Usual average plasma levels should be maintained within 0.1-1000 μM. In cases of local administration or selective uptake, the effective local concentration of the compound cannot be related to plasma concentration.

Compounds provided by the present invention are useful the treatment or prevention of a plurality of diseases and disorders caused by or associated with SSAO activity or inappropriate activity or expression thereof. Particular disorders include inflammatory diseases, adipocyte dysfunction related diseases, carbohydrate metabolism related diseases, vascular diseases, neurodegenerative diseases or cancer. Said diseases and disorders include but are not limited to inflammatory disease including rheumatoid arthritis, osteoarthritis, spondylitis, bone resorption diseases, sepsis, septic shock, atherosclerosis, retinopathy, including diabetic retinopathy, diabetes, chronic pulmonary inflammatory disease, fever, periodontal diseases, ulcerative colitis, pyresis, Alzheimer's and Parkinson's diseases, cystic fibrosis, dysfunctions of the immune system, diabetes onset and maintenance of pancreatic function in diabetes. Preferred diseases and disorders include stroke, multiple sclerosis, migraine, cancer, pain. Generally, the compounds of the invention are provided to advantageously be used for treating or preventing inflammatory eye conditions including uveitis, glaucoma and conjunctivitis; degenerative bone or joint conditions including osteoarthritis, rheumatoid arthritis, rheumatoid spondylitis, gouty arthritis ankylosing spondylitis, psoriatic arthritis and other arthritic conditions, as well as inflamed joints; chronic inflammatory skin conditions, including allergic lesions, lichen planus, pityriasis rosea, eczema, psoriasis, and dermatitis; diseases and disorders of the gastrointestinal tract, including inflammatory bowel disease, Crohn's disease, atrophic gastritis, gastritis varialoforme, ulcerative colitis, coeliac disease, regional ileitis, peptic ulceration, particularly irritable bowel syndrome, reflux oesophagitis, and damage to the gastrointestinal tract resulting from infections, for example, by Helicobacter pylori, or from treatments with non-steroidal anti-inflammatory drugs; inflammatory lung disorders such as asthma, bronchitis, particularly chronic obstructive pulmonary disease, farmer's lung, acute respiratory distress syndrome; bacteraemia, endotoxaemia (septic shock), aphthous ulcers, gingivitis, pyresis, particularly pain, including inflammatory pain, neuropathic pain, acute pain or pain of a central origin; meningitis and pancreatitis; and other conditions associated with inflammation; central nervous system inflammatory conditions and diseases, including multiple sclerosis, Alzheimer's disease, and ischaemia-reperfusion injury associated with ischemic stroke; vascular diseases, such as atheromatous and nonatheromatous arteriosclerosis, ischemic heart disease, and Raynaud's Disease and Phenomenon; diabetes and its complications such as microvascular and macrovascular diseases such as atherosclerosis, vascular retinopathies, nephropathies and neuropathies.

Prophylaxis is expected to be particularly relevant to the treatment of persons who have suffered a previous episode of, or are otherwise considered to be at increased risk of, the disease or condition in question. Persons at risk of developing a particular disease or condition generally include those having a family history of the disease or condition, or those who have been identified by genetic testing or screening to be particularly susceptible to developing the disease or condition.

As used herein, the terms “treating” or “treatment” of a disease includes: (1) preventing the disease, i.e. causing the clinical symptoms of the disease not to develop in a mammal that may be exposed to or predisposed to the disease but does not yet experience or display symptoms of the disease, (2) inhibiting the disease, i.e., arresting or reducing the development of the disease or its clinical symptoms, or (3) relieving the disease, i.e., causing regression of the disease or its clinical symptoms.

As used herein, the term “therapeutically effective amount” means the amount of a compound that, when administered to a mammal for treating a disease, is sufficient to effect such treatment for the disease. The “therapeutically effective amount” will vary depending on the compound, the disease and its severity and the age, weight, or other relevant characteristics of the mammal to be treated.

The compounds of the present invention may be prepared by use of known chemical reactions and procedures. Representative methods for synthesizing compounds of the invention are presented below. It is understood that the nature of the substituents required for the desired target compound often determines the preferred method of synthesis. All variable groups of these methods are as described in the generic description if they are not specifically defined below.

A first general method (Method I) for preparing the agents of formula (I) is summarized in Reaction Scheme 1.

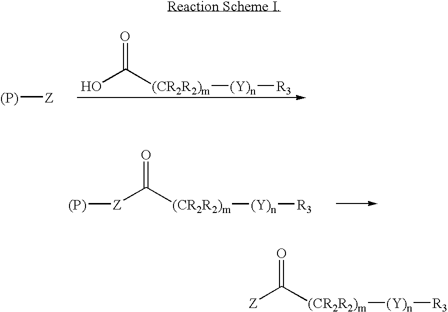

wherein R₁ can be H, C₁-C₆ alkyl, aryl/or substituted aryl, or cycloalkyl, /or where each cycloalkyl group has from 3-7 members, where up to two of the cycloalkyl/members are optionally hetero atoms selected from sulfur, oxygen and nitrogen, and where any member of the alkyl, aryl/or cycloalkyl group is optionally substituted with halogen, C₁-C₆ alkyl or C₁-C₆ alkoxy, aryl or substituted aryl;

wherein R₂ can be H, C₁-C₆ alkyl, aryl/or substituted aryl, C₁-C₆ alkoxyalkyl, or cycloalkyl or cycloalkyl/alkoxy, where each cycloalkyl has from 3-7 members, where up to two of the cycloalkyl members are optionally hetero atoms selected from sulfur, oxygen and nitrogen, and where any member of the alkyl, aryl or cycloalkyl is optionally substituted with halogen, C₁-C₆ alkyl/or C₁-C₆ alkoxy, aryl, halogen, nitro, nitroso, aldehyde, carboxylic acid, —C(O)NH₂, —C(O)NH(C₁-C₆ alkyl), —C(O)N(C₁-C₆ alkyl)(C₁-C₆ alkyl), C₁-C₆ alkoxycarbonyl, sulfate, phosphate, boronic acid, thio, oxime, or imino. Where R₂ is aryl or aryl fused ring (where up to two of the cycloalkyl members are optionally hetero atoms selected from sulfur, oxygen and nitrogen), R₂ can be further substituted in any position with halogen, nitro, nitroso, aldehyde, carboxylic acid, —C(O)NU₂, —C(O)NH(C₁-C₆ alkyl), —C(O)N(C₁-C₆ alkyl)(C₁-C₆ alkyl), amino, C₁-C₆ alkoxycarbonyl, sulfate, phosphate, boronic acid, thio, oxime, imino, or hydroxyl; and

R₃ and Z are as defined in formula I.

This reaction scheme comprises carrying out a condensation with a hydroxylamine group anchored onto a polymeric support (Z=NHR₁O), or Rink Amide (Z=NH₂), or Wang resin (Z=OH), or BAL resin (Z=NHR₁) including the following steps:

a) acylating with the corresponding acid (4 equiv.) the free amino function of the polymeric support using 4 equiv of the corresponding acylating mixture (e.g. HOBt/DIPCDI, HOAt/HATU/DIEA) in 1 mL of DMF at room temperature for 1 h; and

b) releasing the compound of the polymeric support using 1 mL TFA in DCM.

A second general method (Method II) for preparing the agents of formula (I) is summarized in Reaction Scheme 2.

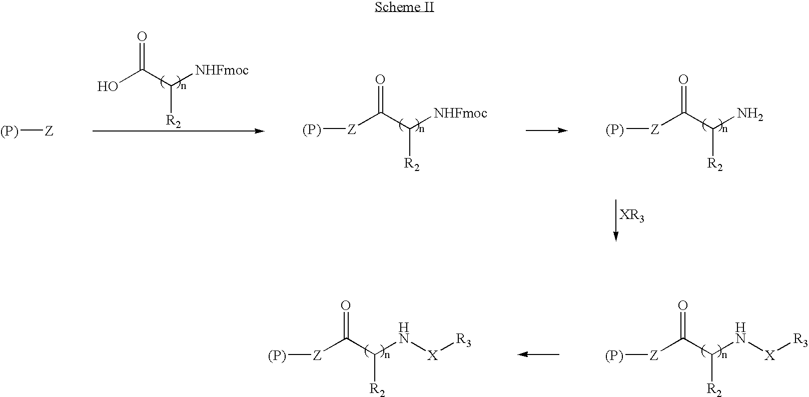

wherein
X can be SO₂ (sulfonyl derivatives), CO (acylated derivates), CH₂ (alkylated derivatives) or CONH (urea derivatives);
R₁ can be C₁-C₆ alkyl, aryl/or substituted aryl, or cycloalkyl/or where each cycloalkyl group has from 3-7 members, where up to two of the cycloalkyl/members are optionally hetero atoms selected from sulfur, oxygen and nitrogen, and where any member of the alkyl, aryl/or cycloalkyl group is optionally substituted with halogen, C₁-C₆ alkyl or C₁-C₆ alkoxy, aryl or substituted aryl;
R₂ can be natural an non natural amino acids side chains;
n is an integer between 1 and 6;
R₃ can be C₁-C₆ alkyl, aryl/or substituted aryl, or cycloalkyl/or cycloalkyl/alkoxy, where each cycloalkyl or aryl group has from 3-7 members, where up to two of the cycloalkyl/members are optionally hetero atoms selected from sulfur, oxygen and nitrogen, and where any member of the alkyl, alkylaryl, alkylaryl fused ring or cycloalkyl/group and alkylaryl is optionally substituted with halogen, C₁-C₆ alkyl/or C₁-C₆ alkoxy, aryl or substituted aryl, arylfused ring, halogen, nitro, nitroso, aldehyde, carboxylic acid, —C(O)N—H₂, —C(O)NH(C₁-C₆ alkyl), —C(O)N(C₁-C₆ alkyl)(C₁-C₆ alkyl), amino, C₁-C₆ alkoxycarbonyl, or sulfate, phosphate, boronic acid, thio, oxime, imino, hydroxyl, or nitrile.
Z is defined in formula I.

The method of scheme 2 comprises carrying out a condensation with a hydroxylamine group anchored onto a polymeric support (Z=NHR₁₀), or Rink Amide (Z=NH₂), or Wang resin (Z=OH), or BAL resin (Z=NHR₁) including the following steps:

a) coupling the amino acid (4 equiv) with the free amino function of the polymeric support using 4 equiv of the corresponding acylating mixture (e.g. HOBt/DIPCDI, HOAt/HATU) in 1 mL of DMF at room temperature for 1 h;

b) removing the protecting group (e.g., 9-fluorenylmethoxycarbonyl) with two treatments of 15 min with 1 ml of piperidine/DMF (50:50) at room temperature;

c1) for sulfonylation: reacting 5 equiv of the corresponding sulfonyl chloride in 1 mL of DCM and 5 equiv. of DIEA at room temperature for 12 h with the free amino function of the amino acid;

c2) for acylation: reacting 3 equiv of the corresponding acid with 3 equiv of HOBt/DIPCDI in 1 mL of DMF as acylating mixture at room temperature for 2 h with the free amino function of the amino acid;

c3) for reductive amination: reacting 5 equiv of the corresponding aldehyde and 5 equiv of NaBH₃CN in 1 mL of AcOH/DMF (1:99) for 3 h at room temperature with the free amino function of the amino acid;

c4) for urea formation (two steps procedure): (a) carbamate formation by reacting 5 equiv of 4-nitrophenil chloroformate and 5 equiv of DIEA in 1 mL of DCM for 12 h at 60° C. with the free amino function of the amino acid, and (b) urea formation by reacting 5 equiv of amine with 5 equiv of TEA in 1 mL of NMP for 12 h at 60° C.

d) releasing the compound of the polymeric support in acidic conditions using 1 mL of TFA in DCM.

A third general method (Method III) for preparing the agents of formula (I) is summarized in Reaction Scheme 3.

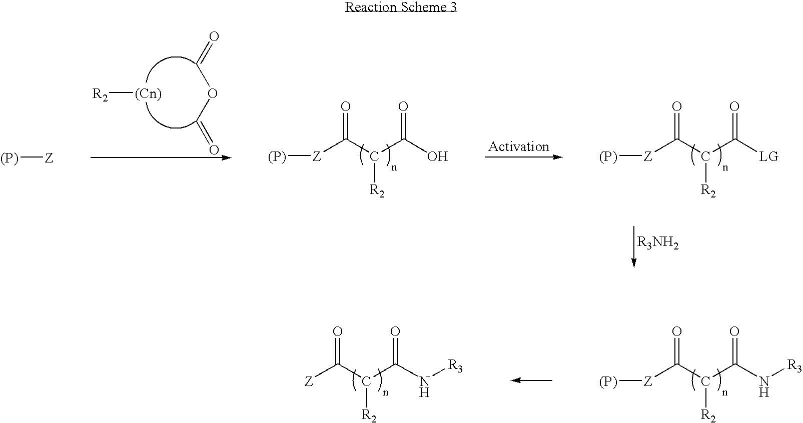

wherein
R₁ can be C₁-C₆ alkyl, aryl/or substituted aryl, or cycloalkyl/or where each cycloalkyl group has from 3-7 members, where up to two of the cycloalkyl/members are optionally hetero atoms selected from sulfur, oxygen and nitrogen, and where any member of the alkyl, aryl/or cycloalkyl group is optionally substituted with halogen, C₁-C₆ alkyl or C₁-C₆ alkoxy, aryl or substituted aryl;

wherein n is an integer between 1-6;
R₂ can be H, C₁-C₆ alkyl, aryl/or substituted aryl, biphenyl or cycloalkyl/or where each cycloalkyl group has from 3-7 members, where up to two of the cycloalkyl/members are optionally hetero atoms selected from sulfur, oxygen and nitrogen, and where any member of the alkyl, aryl/or cycloalkyl/group is optionally substituted with halogen, C₁-C₆ alkyl or C₁-C₆ alkoxy, aryl or substituted aryl;
R₃ can be C₁-C₆ alkyl, aryl/or substituted aryl, or cycloalkyl/or cycloalkyl/alkoxy, where each cycloalkyl/group has from 3-7 members, where up to two of the cycloalkyl/members are optionally hetero atoms selected from sulfur, oxygen and nitrogen, and where any member of the alkyl, alkylaryl, alkylaryl fused ring or cycloalkyl/group and alkylaryl is optionally substituted with halogen, C₁-C₆ alkyl/or C₁-C₆ alkoxy, aryl or substituted aryl, arylfused ring, halogen, nitro, nitroso, aldehyde, carboxylic acid, —C(O)NH₂, —C(O)NH(C₁-C₆ alkyl), —C(O)N(C₁-C₆ alkyl)(C₁-C₆ alkyl), C₁-C₆ alkoxycarbonyl, or sulfate, phosphate, boronic acid, thio, oxime, imino, hydroxyl, or nitrile.
Z is defined in formula I.

The method of Scheme 3 comprises carrying out a condensation with a hydroxylamine group anchored onto a polymeric support (Z=NHR₁₀), or Rink Amide (Z=NH₂), or Wang resin (Z=OH), or BAL resin (Z=NHR₁) including the following steps:

a) overnight acylation of the free amino function of the polymeric support with 5 equiv of the corresponding anhydride in 1 mL of THF at 60° C.;

b) free acid activation with 25 equiv of CDI in 1 mL of DMF for 30 min;

c) coupling the corresponding amine with 3 equiv of HOBt in 1 mL of DMF for 2 h at room temperature; and

d) releasing the compound from the polymeric support in acidic conditions using 1 mL of % TFA in DCM.

A fourth general method (Method IV) for preparing the agents of formula (I) in solution phase is summarized in Reaction Scheme IV.

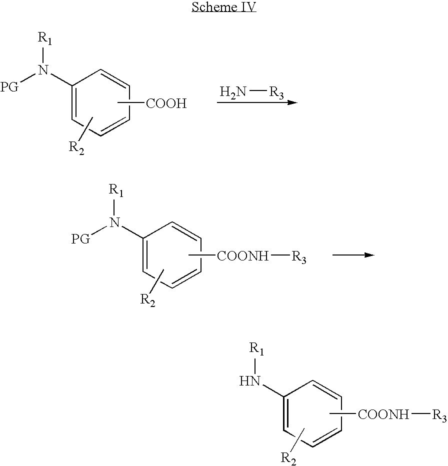

wherein
R₁ can be C₁-C₆ alkyl, aryl/or substituted aryl, or cycloalkyl/or where each cycloalkylgroup has from 3-7 members, where up to two of the cycloalkyl/members are optionally hetero atoms selected from sulfur, oxygen and nitrogen, and where any member of the alkyl, aryl/or cycloalkyl group is optionally substituted with halogen, C₁-C₆ alkyl or C₁-C₆ alkoxy, aryl or substituted aryl;

wherein n is an integer between 1-6;
R₂ can be H, C₁-C₆ alkyl, aryl/or substituted aryl, or cycloalkyl/or cycloalkyl/alkoxy, where each cycloalkyl/group has from 3-7 members, where up to two of the cycloalkyl/members are optionally hetero atoms selected from sulfur, oxygen and nitrogen, and where any member of the alkyl, alkylaryl, alkylaryl fused ring or cycloalkyl/group and alkylaryl is optionally substituted with halogen, C₁-C₆ alkyl/or C₁-C₆ alkoxy, aryl or substituted aryl, arylfused ring, halogen, nitro, nitroso, aldehyde, carboxylic acid, —C(O)NH₂, —C(O)NH(C₁-C₆ alkyl), —C(O)N(C₁-C₆ alkyl)(C₁-C₆ alkyl), C₁-C₆ alkoxycarbonyl, or sulfate, phosphate, boronic acid, thio, oxime, imino, hydroxyl, or nitrile.
R₃ can be C₁-C₆ alkyl, aryl/or substituted aryl, or cycloalkyl/or where each cycloalkyl group has from 3-7 members, where up to two of the cycloalkyl/members are optionally hetero atoms selected from sulfur, oxygen and nitrogen, and where any member of the alkyl, aryl/or cycloalkyl group is optionally substituted with halogen, C₁-C₆ alkyl or C₁-C₆ alkoxy, aryl or substituted aryl; and
PG is Boc, Alloc, F-moc, Bz or any suitable protecting group;

The method of Scheme 4 comprises carrying out a condensation between an amine and carboxylic acid in solution phase, including the following steps:

a) reacting 1 equiv of the corresponding acid with 3 equiv of R₃NH₂/HOBt/DIPCDI in 1.5 mL of DMF as acylating mixture at room temperature for 2 h at room temperature;

b) drying and purification by normal phase, ISOLUTE HM-N 3.0 cartridge or DIAION HP-20;

b) releasing the protecting group following standard procedures (e.g. acidic conditions using 1 mL of HCl/Dioxane 4 M for Boc protecting groups);

The following abbreviations are used herein:
ACN, acetonitrile;
Alloc, allyloxycarbonyl
Boc, t-butyloxycarbonyl
Bz, benzyl
TFA, trifluoroacetic acid;
THF, tetrahydrofurane;
MeOH, methanol;
F-moc, 9-fluorenylmethyloxycarbonyl;
DMF, dimethylformamide;
DCM, methylenechlorode;

### DIEA, N,N-diisopropylethylamine;

CDI, 1,1′-carbonyldiimidazole;
HOBt, 1-hydroxybenzotriazole;
HOAt, 1-Hydroxy-7-azabentriazole;
DIPCDI, N,N′-diisopropylcarbodiimide;
HATU, (N-dimethylamino)-1H-1,2,3-triazolo(4,5-b)pyridine-1-ylmethylene)-N-ethylmethanominium hexafluorophosphate N-oxide;
Cl-Trt, chlorotrityl resin
ESI-MS, Electrospray ionization mass spectroscopy;
IR, infrared spectroscopy;
HPLC, high performance liquid chromatography;
t_{R}, retention time;
NMR, nuclear magnetic resonance;
LG; leaving group;

### PG; Protecting Group; and

### NMP, N-methylpyrrolidone.

Solid-phase manipulations were performed in polypropylene syringes fitted with a polyethylene porous disc. Solvents and soluble reagents were removed by filtration.

Representative compounds prepared according to the methods of the present invention include, but are not limited to the compounds disclosed herein and their pharmaceutically acceptable acid and base addition salts. In addition, if a compound is obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds.

The disclosures in this application of all articles and references, including patents, are incorporated herein by reference.

The following Examples are provided for the purposes of illustration and are not intended to limit the scope of the present invention. The present invention is not to be limited in scope by the exemplified embodiments, which are intended as illustrations of individual aspects of the invention. Indeed, various modifications of the invention in addition to those shown and described herein will become apparent to those skilled in the art from the foregoing description and accompanying drawings. Such modifications are intended to fall within the scope of the appended claims.

### EXAMPLES

### Compounds Obtained by Method I

General Experimental Procedure. Hydroxylamine Cl-Trt resin (100 mg, 0.8 mmol/gr) was acylated with carboxylic acid (4 equiv) and 4 equi. of the acylating mixture HATU/HOBT/DIEA (1:1:2) in DMF (1 mL) as for 1 h at room temperature. The resin was filtered off and washed with 1 mL of DMF (5×1 min) and 1 mL of DCM (5×1 min). The corresponding hydroxamic acid was cleaved with 1 mL 5% of TFA in DCM (3×1 min) and dried. The crude material was dissolved in water and was purified using Diaion HP-20 (500 mg) following standard procedure. HPLC analyses were performed using the following eluent solutions: H₂O (0.1% HCOOH)/ACN(0.07% HCOOH) in a gradient from 0%-100% ACN over 10 min., using an X-Terra C₁₈ 5 μm column (4.6×100) and interrogating the column fractions spectrophotometrically (λ=220/254 nm).

### DIAION HP-20 Purification.

(a) 500 mg of DIAION HP-20 in polypropylene syringes fitted with a polyethylene porous disc were conditioned with three fractions of 10 mL MeOH and with three fractions of 10 mL water;
(b) the crude material was dissolved in 5 mL water and the aqueous fraction was eluted trough the resin;
(c) the resin was washed with 3 fractions of 10 mL water;
(d) the product was eluted with three fractions of 10 mL ACN and the solvent was evaporated.

Table of compounds obtained by Method

| | | Mol. | % HPLC | |
|---|---|---|---|---|
| Ref. N⁰ | Structure | Weight | Purity | MS exp |
| 2 | 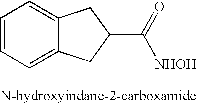 | 177 | 80 | 178.1 |

### Compounds Obtained by Method II

### A. Sulfonylated Hydroxamic Acid Derivative Compounds

General Experimental Procedure. Hydroxylamine Cl-Trt resin (100 mg, 0.8 mmol/gr) was acylated with Fmoc amino acid (4 equivalents) and 4 equiv. of the acylating mixture HATU/HOBT/DIEA (1:1:2) in 1 mL of DMF for 1 h at room temperature. The Fmoc group was then removed by treating with 1 mL of 50% piperidine in DMF (three times for 10 min apiece), and the free amine was sulfonylated overnight with biphenylsulfonyl chloride (5 equiv) in 1 mL of DCM. The resin was filtered off and washed with 1 mL of DCM (5 washes for 1 min apiece) and the extent of the reaction was checked by the ninhydrin test. The corresponding product was cleaved with 1 mL of 5% TFA in DCM (3 treatments for 1 min apiece) and dried. The crude material was dissolved in water and was purified using Diaion HP-20 (500 mg) following standard procedures as disclosed above. The acetonitrile fraction was dried under vacuum. HPLC analyses were performed using the following eluent solutions: H₂O (0.1% HCOOH)/ACN(0.07% HCOOH) in a gradient from 0%-100% ACNover 10 min., using an X-Terra C₁₈ 5 μm column (4.6×100) and interrogating the column fractions spectrophotometrically (k=220/254 nm).

### B. Acylated Hydroxamic Acid Derivatives Compounds.

General Experimental Procedure. Hydroxylamine Cl-Trt resin (100 mg, 0.8 mmol/gr) was acylated with Fmoc amino acid (4 equiv.) and 4 equiv. of the acylating mixture HATU/HOBT/DIEA (1:1:2) in 1 mL of DMF for 1 h at room temperature. The Fmoc group was then removed by treating with 50% piperidine in 1 mL of DMF (10×3 min.), and the free amine was acylated with the corresponding carboxylic acid (3 equiv.) and 3 equiv. of the acylating mixture HOBT/DIPCDI (1:1) in 1 mL of DMF for 2 h at room temperature. The resin was filtered off and washed with 1 mL of DCM (5×1 min) and the extent of the reaction was determined using the ninhydrin test (Kaiser et al., 1970, Analytical Biochem., 34:595-598). The corresponding product was cleaved with 1 mL of 5% TFA in DCM (3×1 min) and dried. The crude material was dissolved in water and was purified using Diaion HP-20 (500 mg) following standard procedures as disclosed above. The acetonitrile fraction was dried under vacuum. HPLC analyses were performed using the following eluent solutions: H₂O (0.1% HCOOH)/ACN(0.07% HCOOH) in a gradient from 0%-100% ACN over 10 min., using an X-Terra C₁₈ 5 μm column (4.6×100) and interrogating the column fractions spectrophotometrically (λ=220/254 nm).

Table of compounds obtained by Method II

| Ref. | | | % HPLC | |
|---|---|---|---|---|
| N⁰ | Structure | Mol. Weight | Purity | MS exp |
| 3 | 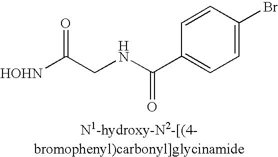 | 272 | 90 | 273.8 |
| 4 | 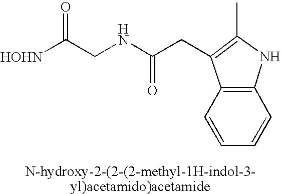 | 260 | 83 | 261 |
| 5 | 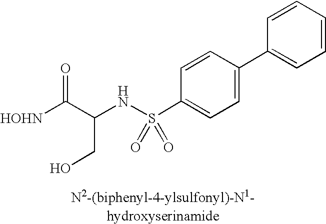 | 336 | 81 | 337.1 |
| 6 | 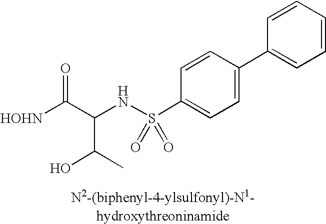 | 350 | 70 | 351.1 |
| 7 | 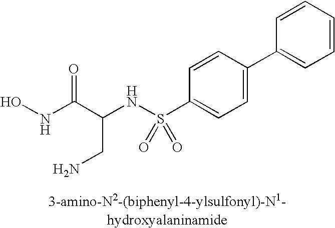 | 335 | 84 | 335 |
| 8 | 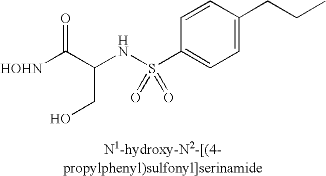 | 302 | 71 | 303.1 |
| 9 | 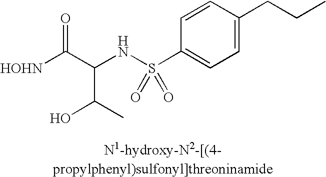 | 316 | 66 | 317.2 |
| 10 | 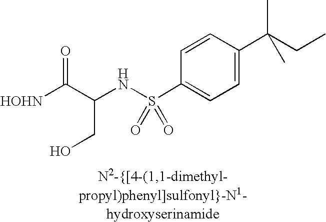 | 330 | 62 | 331.2 |
| 11 | 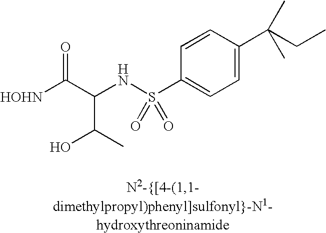 | 344 | 68 | 345 |
| 12 | 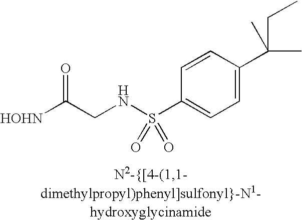 | 300 | 81 | 300.1 |
| 13 | 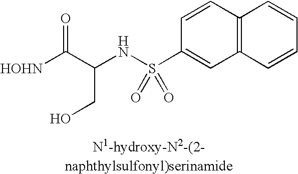 | 310 | 71 | 311.1 |
| 14 | 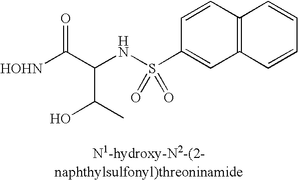 | 324 | 86 | 325.2 |
| 15 | 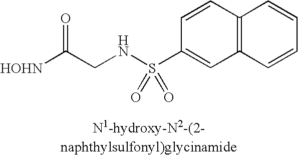 | 280 | 80 | 281 |
| 16 | 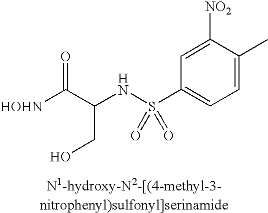 | 310 | 77 | 311.1 |
| 17 | 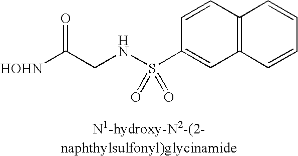 | 280 | 80 | 281 |
| 18 | 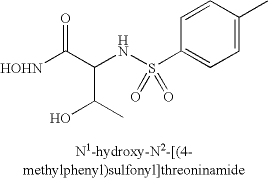 | 287 | 85 | 289.1 |
| 19 | 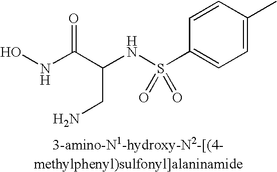 | 273 | 95 | 273 |
| 20 | 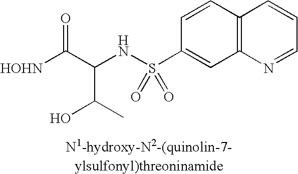 | 325 | 70 | 326.6 |
| 21 | 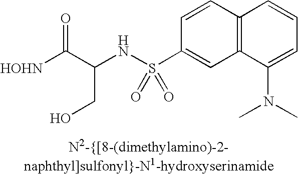 | 352 | 60 | 353.1 |
| 22 | 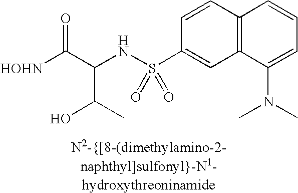 | 327 | 68 | 368.1 |
| 23 | 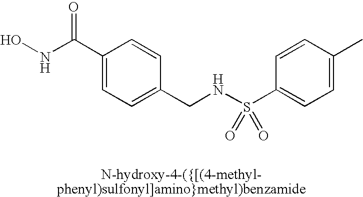 | 320 | 63 | 321 |
| 24 | 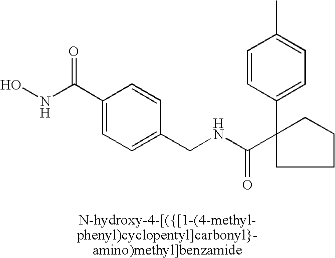 | 352 | 78 | 353.1 |
| 25 | 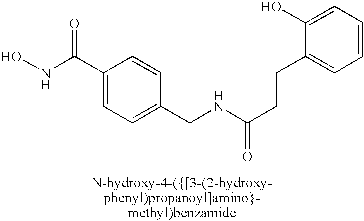 | 314 | 71 | 315.1 |
| 26 | 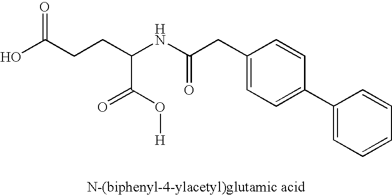 | 341 | 70 | 293.1 |
| 27 | 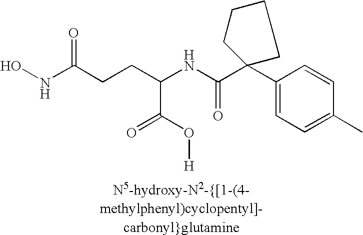 | 348 | 70 | 351.2 |
| 28 | 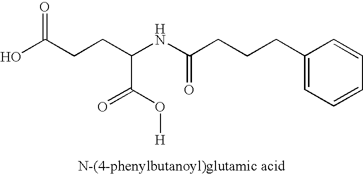 | 293 | 83 | 294.1 |
| 29 | 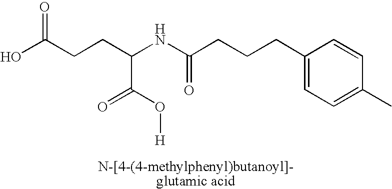 | 307 | 73 | 308.2 |
| 30 | 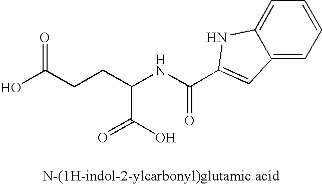 | 290 | 51 | 291.2 |
| 31 | 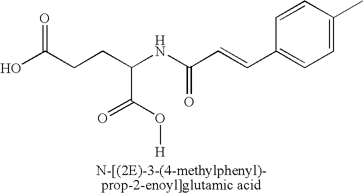 | 291 | 74 | 292.1 |
| 32 | 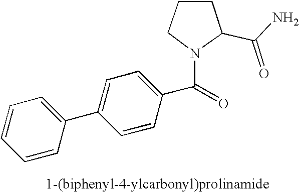 | 294 | 87 | 286.1 |
| 33 | 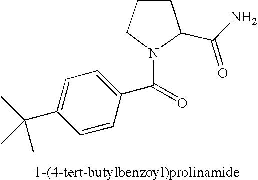 | 274 | 95 | 270.1 |
| 34 | 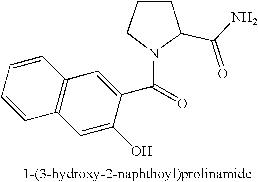 | 284 | 90 | 286.1 |
| 35 | 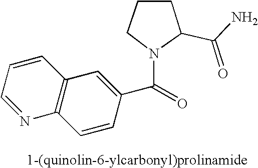 | 269 | 94 | 270.1 |
| 36 | 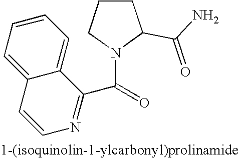 | 269 | 90 | 271 |

### C. Compounds Obtained by Method III

General Experimental Procedure. Hydroxylamine Cl-Trt resin (100 mg, 0.8 mmol/gr) was acylated overnight with succinic anhydride (5 equiv) in THF at 60° C. The resin was filtered off and washed with 1 mL of THF (5×1 min) and 1 mL of DCM (5×1 min) and the extent of the reaction was determined by the ninhydrin test as disclosed above. The carboxyl group was then activated with DIC (25 equiv) in 1 mL of DMF for 30 min. at 25° C. The resin was filtered off and washed with 1 mL of DMF (5×1 min). Afterwards, the corresponding amine (3 equiv) and HOBt (3 equiv) in 1 mL of DMF were added and the mixture was stirred for 2 h at room temperature. The resin was filtered off and washed with 1 mL of THF (5×1 min) and 1 mL of DCM (5×1 min), and the extent of the reaction was checked by the malachite green test (Attardi et al., 2000, Tetrahedron Lett., 41: 7391-7394). The corresponding succinic derivative was cleaved with 1 mL of 5% TFA in DC (3×1 min) and dried. The crude material was dissolved in water and was purified using Diaion HP-20 (500 mg) following standard procedures as described above. The acetonitrile fraction was dried under vacuum. HPLC analyses, which were performed using the following eluent solutions: H₂O (0.1% HCOOH)/ACN(0.07% HCOOH) in a gradient from 0%-100% ACN over 10 min., using an X-Terra C₁₈ 5 μm column (4.6×100) and interrogating the column fractions spectrophotometrically (λ=220/254 nm).

Table of compounds obtained by Method III

| Ref. | | Mol. | | |
|---|---|---|---|---|
| N⁰ | Structure | Weight | % HPLC Purity | MS exp |
| 37 | 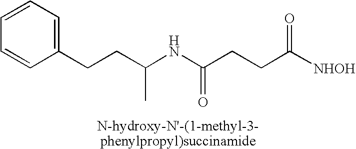 | 264 | 80 | 265.3 |
| 38 | 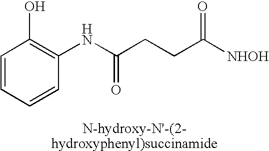 | 224 | 85 | 225.1 |
| 39 | 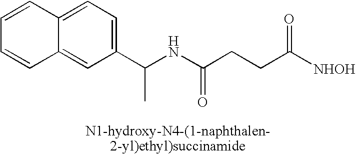 | 286 | 86 | 287.2 |

### D. Compounds Obtained by Method IV

### General Experimental Procedure.

A mixture of 4-tert-Butoxycarbonylamino-3-hydroxy-benzoic acid (1 equiv), HOBT (1 equiv), 1 equiv of the corresponding amine R₃NH₂ and 1 equiv DIPCDI was stirring in DMF (1.5 mL) overnight at room temperature. DMF was eliminated at low pressure and the crude and was purified using the ISOLUTE HM-N 3.0 cartridge and DIAION HP-20 following the standard procedures. Boc protecting group was removal with 1 mL of HCl/Dioxane 4.0 M treatment for a 1 h at room temperature. HPLC analyses were performed using the following eluent solutions: H₂O (0.1% HCOOH)/ACN(0.07% HCOOH) in a gradient from 0%-100% ACN over 10 min., using an X-Terra C₁₈ 5 μm column (4.6×100) and interrogating the column fractions spectrophotometrically (λ=220/254 nm).

### ISOLUTE HM-N 3.0 Cartridge

(a) crude material was dissolved in 1.5 mL of 5% NaHCO₃ and 1.5 mL of ethyl acetate and was eluted trough the cartridge;
(b) cartridge was washed with 3 fractions of 4 mL of ethyl acetate the solvent was evaporated.

Table of compounds obtained by Method IV

| ref | | | | |
|---|---|---|---|---|
| n⁰ | structure | Molec Wt. | HPLC | MS exp |
| 40 | 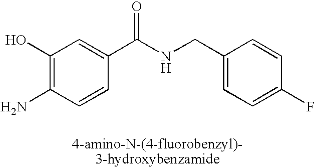 | 260 | 93 | 261.2 |
| 41 | 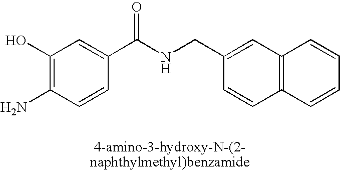 | 292 | 81 | 293.1 |
| 42 | 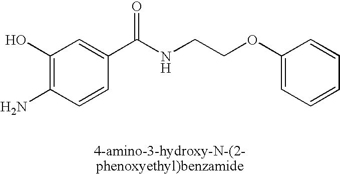 | 272 | 91 | 272 |
| 43 | 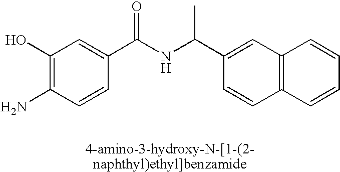 | 306 | 96 | 307.2 |
| 44 | 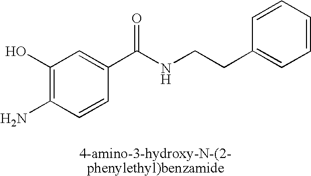 | 256 | 94 | 257.2 |
| 45 | 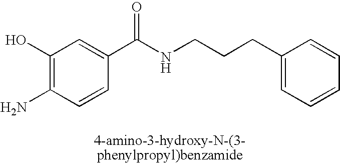 | 270 | 82 | 271.3 |
| 46 | 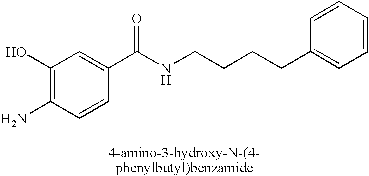 | 284 | 93 | 285.1 |
| 47 | 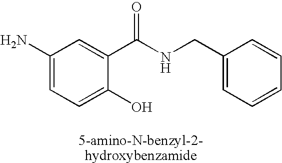 | 242 | 98 | 243 |
| 48 | 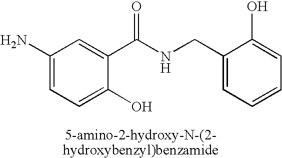 | 258 | 98 | 257 |
| 49 | 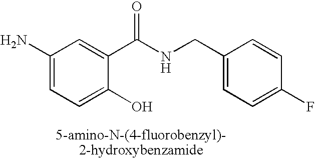 | 260 | 92 | 260 |
| 50 | 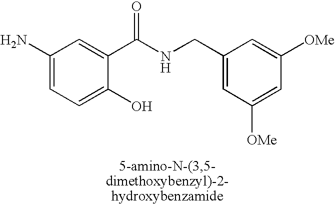 | 240 | 97 | 302 |
| 51 | 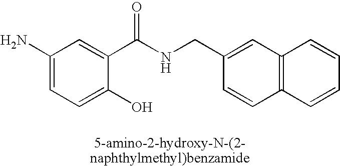 | 292 | 93 | 284 |
| 52 | 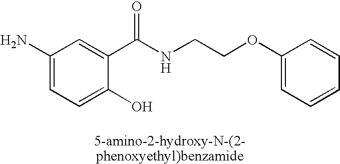 | 272 | 97 | 272 |
| 53 | 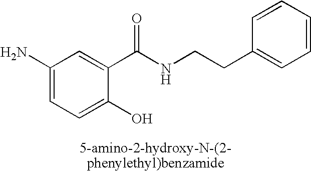 | 256 | 97 | 256 |
| 54 | 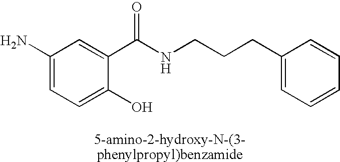 | 270 | 98 | 270 |
| 55 | 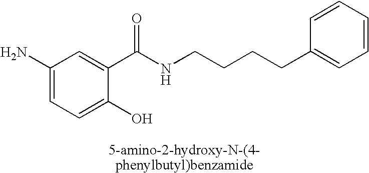 | 284 | 92 | 284 |

The following compounds are prepared essentially according to the methods and procedures described above.

### Name

N¹-hydroxy-N²-[2-(4-methylphenyl)propanoyl]serinamide
N¹-hydroxy-N²-[2-(4-methylphenyl)propanoyl]threoninamide
N¹-hydroxy-N²-[2-(4-methylphenyl)propanoyl]cysteinamide
3-amino-N¹-hydroxy-N²-[2-(4-methylphenyl)propanoyl]alaninamide
N-((hydroxycarbamoyl)methyl)-2-p-tolylpropanamide
2-(biphenyl-4-ylacetyl)-N¹-hydroxyserinamide
N²-(biphenyl-4-ylacetyl)-N¹-hydroxyserinamide
N²-(biphenyl-4-ylacetyl)-N¹-hydroxycysteinamide
3-amino-N²-(biphenyl-4-ylacetyl)-N¹-hydroxyalaninamide
N²-(biphenyl-4-ylacetyl)-N¹-hydroxyglycinamide
N-(1-(hydroxycarbamoyl)-2-hydroxyethyl)-1-p-tolylcyclopentanecarboxamide
N-(1-(hydroxycarbarnoyl)-2-hydroxypropyl)-1-p-tolylcyclopentanecarboxamide
N-(1-(hydroxycarbamoyl)-2-mercaptoethyl)-1-p-tolylcyclopentanecarboxamide
N-(1-(hydroxycarbamoyl)-2-aminoethyl)-1-p-tolylcyclopentanecarboxamide
N-((hydroxycarbamoyl)methyl)-1-p-tolylcyclopentanecarboxamide
N¹-hydroxy-N²-[3-(2-hydroxyphenyl)propanoyl]serinamide
N¹-hydroxy-N²-[3-(2-hydroxyphenyl)propanoyl]threoninamide
N-hydroxy-N²-[3-(2-hydroxyphenyl)propanoyl]cysteinamide
3-amino-1-hydroxy-N²-[3-(2-hydroxyphenyl)propanoyl]alaninamide
N-((hydroxycarbamoyl)methyl)-3-(2-hydroxyphenyl)propanamide
N-(1-(hydroxycarbamoyl)-2-hydroxyethyl)-4-phenylbutanamide
N¹-hydroxy-N²-(4-phenylbutanoyl)threoninamide
N-(1-(hydroxycarbamoyl)-2-mercaptoethyl)-4-phenylbutanamide
N-(1-(hydroxycarbamoyl)-2-aminoethyl)-4-phenylbutanamide
N-((hydroxycarbamoyl)methyl)-4-phenylbutanamide
N-(1-(hydroxycarbamoyl)-2-hydroxyethyl)-4-p-tolylbutanamide
N¹-hydroxy-N²-[4-(4-methylphenyl)butanoyl]threoninamide
N-(1-(hydroxycarbamoyl)-2-mercaptoethyl)-4-p-tolylbutanamide
N-(1-(hydroxycarbamoyl)-2-aminoethyl)-4-p-tolylbutanamide
N-((hydroxycarbamoyl)methyl)-4-p-tolylbutanamide
(E)-N-(1-(hydroxycarbamoyl)-2-hydroxyethyl)-3-(furan-2-yl)acrylamide
2-((E)-3-(furan-3-yl)acrylamido)-N,3-dihydroxybutanamide
(E)-N-(1-(hydroxycarbaroyl)-2-mercaptoethyl)-3)-(furan-2-yl)-2-methylacrylamide
3-amino-N²-[(2E)-3-(2-furyl)prop-2-enoyl]-N-hydroxyalaninamide
(E)-N-((hydroxycarbamoyl)methyl)-3-(furan-2-yl)acrylamide
N-(1-(hydroxycarbamoyl)-2-hydroxyethyl)-1H-indole-3-carboxamide
N-(1-(hydroxycarbamoyl)-2-hydroxypropyl)-1H-indole-3-carboxamide
N-(1-(hydroxycarbamoyl)-2-mercaptoethyl)-1H-indole-3-carboxamide
N-(1-(hydroxycarbamoyl)-2-aminoethyl)-1H-indole-3-carboxamide
N-((hydroxycarbamoyl)methyl)-1H-indole-3-carboxamide
N-(1-(hydroxycarbamoyl)-2-hydroxyethyl)-1H-indole-2-carboxamide
N-(1-(hydroxycarbamoyl)-2-hydroxypropyl)-1H-indole-2-carboxamide
N-(1-(hydroxycarbamoyl)-2-hydroxyethyl)-1H-indole-2-carboxamide
N-(1-(hydroxycarbamoyl)-2-aminoethyl)-1H-indole-2-carboxamide
N-((hydroxycarbamnoyl)methyl)-1H-indole-2-carboxamide
(E)-N-(1-(hydroxycarbamoyl)-2-hydroxyethyl)cinnamamide
2-(cinnamamido)-N,3-dihydroxybutanamide
(E)-N-(1-(hydroxycarbamoyl)-2-mercaptoethyl)cinnamamide
(E)-N-(1-(hydroxycarbamoyl)-2-aminoethyl)cinnamamide
(E)-N-((hydroxycarbamoyl)methyl)cinnamamide;
N¹-hydroxy-N²-(2-hydroxy-3-nitrobenzyl)serinamide
N¹-hydroxy-N²-(2-hydroxy-3-nitrobenzyl)threoninamide
N¹-hydroxy-N²-(2-hydroxy-3-nitrobenzyl)cysteinamide
3-amino-N¹-hydroxy-N²-(2-hydroxy-3-nitrobenzyl)alaninamide
N¹-hydroxy-N²-(2-hydroxy-3-nitrobenzyl)glycinamide
N¹-hydroxy-N²-[(4-methoxy-2-naphthyl)methyl]serinamide
N¹-hydroxy-N²-[(4-methyl-2-naphthyl)methyl]threoninamide-methanol (1:1)
N¹-hydroxy-N²-[(4-methoxy-2-naphthyl)methyl]cysteinamide
3-amino-1-hydroxy-2-[(4-methoxy-2-naphthyl)methyl]alaninamide
N¹-hydroxy-N²-[(4-methoxy-2-naphthyl)methyl]glycinamide
N¹-hydroxy-N²-(2-nitrobenzyl)serinamide
N¹-hydroxy-N²-(2-nitrobenzyl)threoninamide
N¹-hydroxy-N²-(2-nitrobenzyl)cysteinamide
3-amino-N¹-hydroxy-N²-(2-nitrobenzyl)alaninamide
N¹-hydroxy-N²-(2-nitrobenzyl)glycinamide
N²-(biphenyl-2-ylmethyl)-N¹-hydroxyserinamide
N²-(biphenyl-2-ylmethyl)-N¹-hydroxythreoninamide
N²-(biphenyl-2-ylmethyl)-N¹-hydroxycysteinamide
3-amino-N²-(biphenyl-2-ylmethyl)-N¹-hydroxyalaninamide
N²-(biphenyl-2-ylmethyl)-N-hydroxyglycinamide
N²-(2-fluoro-6-methoxybenzyl)-N₁-hydroxyserinamide
N²-(2-fluoro-6-methoxybenzyl)-N¹-hydroxythreoninamide
N²-(2-fluoro-6-methoxybenzyl)-N⁴-hydroxycysteinamide
3-amino-N²-(2-fluoro-6-methoxybenzyl)-N¹-hydroxy alaninamide
N²-(2-fluoro-6-methoxybenzyl)-N¹-hydroxyglycinamide
3-hydroxy-N²-(2-hydroxy-6-methoxybenzyl)serinamide
N¹-hydroxy-N²-(2-hydroxy-6-methoxybenzyl)threoninamide
N¹-hydroxy-N²-(2-hydroxy-6-methoxybenzyl)cysteinamide
3-amino-1-hydroxy-N²-(2-hydroxy-6-methoxybenzyl)alaninamide
N¹-hydroxy-N²-(2-hydroxy-6-methoxybenzyl)glycinamide
N¹-hydroxy-N²-[(1-hydroxy-2-naphthyl)methyl]serinamide
N¹-hydroxy-N²-[(1-hydroxy-2-naphthyl)methyl]threoninamide
N¹-hydroxy-N²-[(1-hydroxy-2-naphthyl)methyl]cysteinamide
3-amino-N¹-hydroxy-N²-[(1-hydroxy-2-naphthyl)methyl]alaninamide
N¹-hydroxy-N²-[(1-hydroxy-2-naphthyl)methyl]glycinamide
N²-[4-(benzyloxy)benzyl]-N-hydroxyserinamide
N²-[4-(benzyloxy)benzyl]-N¹-hydroxythreoninamide
N²-[4-(benzyloxy)benzyl]-N²-hydroxycysteinamide
3-amino-N²-[4-(benzyloxy)benzyl]-N¹-hydroxyalaninamide
N²-[4-(benzyloxy)benzyl]-N¹-hydroxyglycinamide
N²-(3-fluoro-4-methylbenzyl)-N¹-hydroxyserinamide
N²-(3-fluoro-4-methylbenzyl)-N¹-hydroxythreoninamide
N²-(3-fluoro-4-methylbenzyl)-N¹-hydroxycysteinamide
3-amino-N²-(3-fluoro-4-methylbenzyl)-N¹-hydroxyalaninamide
N²-(3-fluoro-4-methylbenzyl)-N¹-hydroxyglycinamide
N¹-hydroxy-N²-(2-naphthylmethyl)serinamide
N¹-hydroxy-N²-(2-naphthylmethyl)threoninamide
N¹-hydroxy-N²-(2-naphthylmethyl)cysteinamide
3-amino-N¹-hydroxy-N²-(2-naphthylmethyl)alaninamide
N¹-hydroxy-N²-(2-naphthylmethyl)glycinamide;
N²-(biphenyl-4-ylsulfonyl)-N-hydroxyserinamide
N²-(biphenyl-4-ylsulfonyl)-N¹-hydroxythreoninamide
N²-(biphenyl-4-ylsulfonyl)-N¹-hydroxycysteinamide
3-amino-N²-(biphenyl-4-ylsulfonyl)-N¹-hydroxyalaninamide
N²-(biphenyl-4-ylsulfonyl)-N¹-hydroxyglycinamide
N¹-hydroxy-N²-[(4-propylphenyl)sulfonyl]serinamide
N¹-hydroxy-N²-[(4-propylphenyl)sulfonyl]threoninamide
N¹-hydroxy-N²-[(4-propylphenyl)sulfonyl]cysteinamide
3-amino-N¹-hydroxy-N²-[(4-propylphenyl)sulfonyl]alaninamide
N¹-hydroxy-N²-[(4-propylphenyl)sulfonyl]glycinamide
N²-{[4-(1,1-dimethylpropyl)phenyl]sulfonyl}-N¹-hydroxyserinamide
N²-{[4-(1,1-dimethylpropyl)phenyl]sulfonyl}-N¹-hydroxythreoninamide
N²-{[4-(1,1-dimethylpropyl)phenyl]sulfonyl}-N¹-hydroxycysteinamide
3-amino-N²-{[4-(1,1-dimethylpropyl)phenyl]sulfonyl}-N¹-hydroxyalaninamide
N²-{[4-(1,1-dimethylpropyl)phenyl]sulfonyl}-N¹-hydroxyglycinamide
N¹-hydroxy-N²-(2-naphthylsulfonyl)serinamide
N¹-hydroxy-N²-(2-naphthylsulfonyl)threoninamide
N¹-hydroxy-N²-(2-naphthylsulfonyl)cysteinamide
3-amino-N¹-hydroxy-N²-(2-naphthyl sulfonyl)alaninamide
N¹-hydroxy-N²-(2-naphthylsulfonyl)glycinamide
N¹-hydroxy-N²-[(4-methyl)sulfonyl]serinamide
N¹-hydroxy-N²-[(4-methyl-3-nitrophenyl)sulfonyl]threoninamide
N¹-hydroxy-N²-[(4-methyl-3-nitrophenyl)sulfonyl]cysteinamide
3-amino-N¹-hydroxy-N²-[(4-methyl-3-nitrophenyl)sulfonyl]alaninamide
N¹-hydroxy-N²-[(4-methyl)sulfonyl]glycinamide
N¹-hydroxy-N²-[(4-methylphenyl)sulfonyl]serinamide
N¹-hydroxy-N²-[(4-methylphenyl)sulfonyl]serinamide
N¹-hydroxy-N²-[(4-methylphenyl)sulfonyl]cysteinamide
3-amino-N¹-hydroxy-N²-[(4-methylphenyl)sulfonyl]alaninamide
N¹-hydroxy-N²-[(4-methylphenyl)sulfonyl]glycinamide
N¹-hydroxy-N²-{[4-(trifluoromethyl)phenyl]sulfonyl}serinamide
N¹-hydroxy-N²-{[4-(trifluoromethyl)phenyl]sulfonyl}serinamide
N¹-hydroxy-N²-{[4-(trifluoromethyl)phenyl]sulfonyl}cysteinamide
3-amino-N¹-hydroxy-N²-{[4-(trifluoromethyl)phenyl]sulfonyl}alaninamide
N¹-hydroxy-N²-{[4-(trifluoromethyl)phenyl]sulfonyl}glycinamide
N¹-hydroxy-N²-{[2-nitro-4-(trifluoromethyl)phenyl]sulfonyl}serinamide
N¹-hydroxy-N²-{[2-nitro-4-(trifluoromethyl)phenyl]sulfonyl}threoninamide
N¹-hydroxy-N²-{[2-nitro-4-(trifluoromethyl)phenyl]sulfonyl}cysteinamide
3-amino-N¹-hydroxy-N²-{[2-nitro-4-(trifluoromethyl)phenyl]sulfonyl}alaninamide
N¹-hydroxy-N²-{[2-nitro-4-(trifluoromethyl)phenyl]sulfonyl}glycinamide
N¹-hydroxy-N²-(quinolin-7-ylsulfonyl)serinamide
N¹-hydroxy-N²-(quinolin-7-ylsulfonyl)threoninamide
N¹-hydroxy-N²-(quinolin-7-ylsulfonyl)cysteinamide
3-amino-N¹-hydroxy-N²-(quinolin-7-ylsulfonyl)alaninamide
N¹-hydroxy-N²-(quinolin-7-ylsulfonyl)glycinamide
N²-{[8-(dimethylamino)-2-naphthyl]sulfonyl}-N¹-hydroxyserinamide
N²-{[8-(dimethylamino)-2-naphthyl]sulfonyl}-N¹-hydroxythreoninamide
N²-{[8-(dimethylamino)-2-naphthyl]sulfonyl}-N¹-hydroxycysteinamide
3-amino-N²-{[8-(dimethylamino)-2-naphthyl]sulfonyl}-N¹-hydroxyalaninamide
N²-{[8-(dimethylamino)-2-naphthyl]sulfonyl}-N¹-hydroxyglycinamide;
N²-(biphenyl-4-ylsulfonyl)-N¹-hydroxyserinamide
N²-(biphenyl-4-ylsulfonyl)-N¹-hydroxythreoninamide
N²-(biphenyl-4-ylsulfonyl)-N¹-hydroxycysteinamide
3-amino-N²-(biphenyl-4-ylsulfonyl)-M-hydroxyalaninamide
N²-(biphenyl-4-ylsulfonyl)-N¹-hydroxyglycinamide
N¹-hydroxy-N²-[(4-propylphenyl)sulfonyl]serinamide
N¹-hydroxy-N²-[(4-propylphenyl)sulfonyl]threoninamide
N¹-hydroxy-N²-[(4-propylphenyl)sulfonyl]cysteinamide
3-amino-N¹-hydroxy-N²-[(4-propylphenyl)sulfonyl]alaninamide
N¹-hydroxy-N²-[(4-propylphenyl)sulfonyl]glycinamide
N²-{[4-(1,1-dimethylpropyl)phenyl]sulfonyl}-N¹-hydroxyserinamide
N²-{[4-(1,1-dimethylpropyl)phenyl]sulfonyl}-N¹-hydroxythreoninamide
N²-{[4-(1,1-dimethylpropyl)phenyl]sulfonyl}-N¹-hydroxycysteinamide
3-amino-N²-{[4-(1,1-dimethylpropyl)phenyl]sulfonyl}-N¹-hydroxyalaninamide
N2-{[4-(1,1-dimethylpropyl)phenyl]sulfonyl}-N¹-hydroxyglycinamide
N¹-hydroxy-N²-(2-naphthylsulfonyl)serinamide
N¹-hydroxy-N²-(2-naphthylsulfonyl)threoninamide
N¹-hydroxy-N²-(2-naphthylsulfonyl)cysteinamide
3-amino-N¹-hydroxy-N²-(2-naphthylsulfonyl)alaninamide
N¹-hydroxy-N²-(2-naphthylsulfonyl)glycinamide
N¹-hydroxy-N²-[(4-methyl-3-nitrophenyl)sulfonyl]serinamide
N¹-hydroxy-N²-[(4-methyl-3-nitrophenyl)sulfonyl]threoninamide
N¹-hydroxy-N²-[(4-methyl-3-nitrophenyl)sulfonyl]cysteinamide
3-amino-N¹-hydroxy-N²-[(4-methyl-3-nitrophenyl)sulfonyl]alaninamide
N¹-hydroxy-N²-[(4-methyl-3-nitrophenyl)sulfonyl]glycinamide
N¹-hydroxy-N²-[(4-methylphenyl)sulfonyl]serinamide
N¹-hydroxy-N²-[(4-methylphenyl)sulfonyl]threoninamide
N¹-hydroxy-N²-[(4-methylphenyl)sulfonyl]cysteinamide
N¹-hydroxy-N²-[(4-methylphenyl)sulfonyl]glycinamide
N¹-hydroxy-N²-{[4-(trifluoromethyl)phenyl]sulfonyl}serinamide
N¹-hydroxy-N²-{[4-(trifluoromethyl)phenyl]sulfonyl}serinamide
N¹-hydroxy-N²-{[4-(trifluoromethyl)phenyl]sulfonyl}cysteinamide
3-amino-N¹-hydroxy-N²-{[4-(trifluoromethyl)phenyl]sulfonyl}alaninamide
N¹-hydroxy-N²-{[4-(trifluoromethyl)phenyl]sulfonyl}glycinamide
N¹-hydroxy-N²-{[2-nitro-4-(trifluoromethyl)phenyl]sulfonyl}serinamide
N¹-hydroxy-N²-{[2-nitro-4-(trifluoromethyl)phenyl]sulfonyl}threoninamide
N¹-hydroxy-N²-{[2-nitro-4-(trifluoromethyl)phenyl]sulfonyl}cysteinamide
3-amino-N¹-hydroxy-N²-{[2-nitro-4-(trifluoromethyl)phenyl]sulfonyl}alaninamide
N¹-hydroxy-N²-{[2-nitro-4-(trifluoromethyl)phenyl]sulfonyl}glycinamide
N¹-hydroxy-N²-(quinolin-7-ylsulfonyl)serinamide
N¹-hydroxy-N²-(quinolin-7-ylsulfonyl)threoninamide
N¹-hydroxy-N²-(quinolin-7-ylsulfonyl)cysteinamide
3-amino-N¹-hydroxy-N²-(quinolin-7-ylsulfonyl)alaninamide
N¹-hydroxy-N²-(quinolin-7-ylsulfonyl)glycinamide
N²-{[8-(dimethylamino)-2-naphthyl]sulfonyl}-N¹-hydroxyserinamide
N²-{[8-(dimethylamino)-2-naphthyl]sulfonyl}-N¹-hydroxythreoninamide
N²-{[8-(dimethylamino)-2-naphthyl]sulfonyl}-N¹-hydroxycysteinamide
3-amino-N²-{[-(dimethylamino)-2-naphthyl]sulfonyl}-N¹-hydroxyalaninamide
N²-{[8-(dimethylamino)-2-naphthyl]sulfonyl}-N¹-hydroxyglycinamide;
N-hydroxy-N²-(2-hydroxy-5-methylphenyl)phthalamide
N-hydroxy-N′-(2-hydroxy-5-methylphenyl)biphenyl-2,2′-dicarboxamide
N-hydroxy-N′-(2-hydroxy-5-methylphenyl)naphthalene-1,8-dicarboxamide
N-hydroxy-N′-(2-hydroxy-5-methylphenyl)succinamide
N-hydroxy-N′-(2-hydroxy-4-nitrophenyl)phthalamide
N-hydroxy-N′-(2-hydroxy-4-nitrophenyl)biphenyl-2,2′-dicarboxamide
N-hydroxy-N′-(2-hydroxy-4-nitrophenyl)naphthalene-1,8-dicarboxamide
N-hydroxy-N′-(2-hydroxy-4-nitrophenyl)succinamide
N-hydroxy-N′-(2-hydroxy-3-methylphenyl)phthalamide
N-hydroxy-N′-(2-hydroxy-3-methylphenyl)biphenyl-2,2′-dicarboxamide
N-hydroxy-N′-(2-hydroxy-3-methylphenyl)naphthalene-1,8-dicarboxamide
N-hydroxy-N′-(2-hydroxy-3-methylphenyl)succinamide
N-hydroxy-N′-(2-hydroxyphenyl)phthalamide
N-hydroxy-N′-(2-hydroxyphenyl)biphenyl-2,2′-dicarboxamide
N-hydroxy-N′-(2-hydroxyphenyl)naphthalene-1,8-dicarboxamide
N-hydroxy-N′-(2-hydroxyphenyl)succinamide
N-hydroxy-N′-(3-hydroxy-2-naphthyl)phthalamide
N-hydroxy-N′-(3-hydroxy-2-naphthyl)biphenyl-2,2′-dicarboxamide
N-hydroxy-N′-(3-hydroxy-2-naphthyl)naphthalene-1,8-dicarboxamide
N-hydroxy-N′-(3-hydroxy-2-naphthyl)succinamide
N-hydroxy-N′-(2-mercaptophenyl)phthalamide
N-hydroxy-N′-(2-mercaptophenyl)biphenyl-2,2′-dicarboxamide
N-hydroxy-N′-(2-mercaptophenyl)naphthalene-1,8-dicarboxamide
N-hydroxy-N′-(2-mercaptophenyl)succinamide
N-(3-acetylphenyl)-N′-hydroxyphthalimide
N-(3-acetylphenyl)-N′-hydroxybiphenyl-2,2′-dicarboxamide
N-(3-acetylphenyl)-N′-hydroxynaphthalene-1,8-dicarboxamide
N-(3-acetylphenyl)-N′-hydroxysuccinamide
N-hydroxy-N′-(2-nitropyridin-3-yl)phthalamide
N-hydroxy-N′-(2-nitropyridin-3-yl)biphenyl-2,2′-dicarboxamide
N-hydroxy-N′-(2-nitropyridin-3-yl)naphthalene-1,8-dicarboxamide
N-hydroxy-N′-(2-nitropyridin-3-yl)succinamide
N-(2-aminophenyl)-N′-hydroxyphthalimide
N-(2-aminophenyl)-N′-hydroxybiphenyl-2,2′-dicarboxamide
N-(2-aminophenyl)-N′-hydroxynaphthalene-1,8-dicarboxamide
N-(2-aminophenyl)-N′-hydroxysuccinamide
N-(8-amino-1-naphthyl)-N′-hydroxyphthalimide
N-(8-amino-1-naphthyl)-N′-hydroxybiphenyl-2,2′-dicarboxamide
N-(8-amino-1-naphthyl)-N′-hydroxynaphthalene-1,8-dicarboxamide
N-(8-amino-1-naphthyl)-M-hydroxysuccinamide;
2-[(2-hydroxy-4-methylbenzoyl)amino]isonicotinamide
N-[5-(aminocarbonyl)-2-hydroxyphenyl]-2-hydroxy-4-methylbenzamide
2-[(biphenyl-4-ylcarbonyl)amino]isonicotinamide
N-[5-(aminocarbonyl)-2-hydroxyphenyl]biphenyl-4-carboxamide
2-[(4-tert-butylbenzoyl)amino]isonicotinamide
3-[(4-tert-butylbenzoyl)amino]-4-hydroxybenzamide
2-[(1-hydroxy-2-naphthoyl)amino]isonicotinamide
N-[5-(aminocarbonyl)-2-hydroxyphenyl]-1-hydroxy-2-naphthamide
2-[(3-hydroxy-2-naphthoyl)amino]isonicotinamide
N-[5-(aminocarbonyl)-2-hydroxyphenyl]-3-hydroxy-2-naphthamide
N-[4-(aminocarbonyl)pyridin-2-yl]quinoline-6-carboxamide
N-[5-(aminocarbonyl)-2-hydroxyphenyl]quinoline-6-carboxamide
N-[4-(aminocarbonyl)pyridin-2-yl]isoquinoline-3-carboxamide
N-[5-(aminocarbonyl)-2-hydroxyphenyl]isoquinoline-3-carboxamide
N-[4-(aminocarbonyl)pyridin-2-yl]pyridine-2-carboxamide
N-[5-(aminocarbonyl)-2-hydroxyphenyl]pyridine-2-carboxamide
N-[4-(aminocarbonyl)pyridin-2-yl]-2-mercaptonicotinamide
N-[5-(aminocarbonyl)-2-hydroxyphenyl]-2-mercaptonicotinamide
N-[4-(aminocarbonyl)pyridin-2-yl]isoquinoline-1-carboxamide
N-[5-(aminocarbonyl)-2-hydroxyphenyl]isoquinoline-1-carboxamide;
2-({[(2-hydroxy-5-methylphenyl)amino]carbonyl}amino)isonicotinamide
4-hydroxy-3-({[(2-hydroxy-5-methylphenyl)amino]carbonyl}amino)benzamide
2-({[(2-hydroxy-4-nitrophenyl)amino]carbonyl}amino)isonicotinamide
4-hydroxy-3-({[(2-hydroxy-4-nitrophenyl)amino]carbonyl}amino)benzamide
2-({[(2-hydroxy-6-methylphenyl)amino]carbonyl}amino)isonicotinamide
4-hydroxy-3-({[(2-hydroxy-6-methylphenyl)amino]carbonyl}amino)benzamide
2-({[(2-hydroxyphenyl)amino]carbonyl}amino)isonicotinamide
4-hydroxy-3-({[(2-hydroxyphenyl)amino]carbonyl}amino)benzamide
2-({[(3-hydroxy-2-naphthyl)amino]carbonyl}amino)isonicotinamide
4-hydroxy-3-({[(3-hydroxy-2-naphthyl)amino]carbonyl}amino)benzamide
2-({[(2-mercaptophenyl)amino]carbonyl}amino)isonicotinamide
4-hydroxy-3-({[(2-mercaptophenyl)amino]carbonyl}amino)benzamide
2-({[(3-acetylphenyl)amino]carbonyl}amino)isonicotinamide
3-({[(3-acetylphenyl)amino]carbonyl}amino)-4-hydroxybenzamide
2-({[(2-nitropyridin-3-yl)amino]carbonyl}amino)isonicotinamide
4-hydroxy-3-({[(2-nitropyridin-3-yl)amino]carbonyl}amino)benzamide
2-({[(2-aminophenyl)amino]carbonyl}amino)isonicotinamide
3-({[(2-aminophenyl)amino]carbonyl}amino)-4-hydroxybenzamide
2-({[(8-amino-1-naphthyl)amino]carbonyl}amino)isonicotinamide
3-({[(8-amino-1-naphthyl)amino]carbonyl}amino)-4-hydroxybenzamide;
4-{[(biphenyl-4-ylsulfonyl)amino]methyl}-N-hydroxybenzamide
N-hydroxy-4-({[(4-propylphenyl)sulfonyl]amino}methyl)benzamide
4-[({[4-(1,1-dimethylpropyl)phenyl]sulfonyl}amino)methyl]-N-hydroxybenzamide
N-hydroxy-4-{[(2-naphthylsulfonyl)amino]methyl}benzamide
N-hydroxy-4-({[(4-methyl-3-nitrophenyl)sulfonyl]amino}methyl)benzamide
N-hydroxy-4-({[(4-methylphenyl)sulfonyl]amino}methyl)benzamide
N-hydroxy-4-[({[4-(trifluoromethyl)phenyl]sulfonyl}amino)methyl]benzamide
N-hydroxy-4-({[(4-methyl-2-nitrophenyl)sulfonyl]amino}methyl)benzamide
N-hydroxy-4-{[(quinolin-7-ylsulfonyl)amino]methyl}benzamide
4-[({[8-(dimethylamino)-2-naphthyl]sulfonyl}amino)methyl]-N-hydroxybenzamide
N-hydroxy-4-({[2-(4-methylphenyl)propanoyl]amino}methyl)benzamide
4-{[(biphenyl-4-ylacetyl)amino]methyl}-N-hydroxybenzamide
N-hydroxy-4-[({[1-(4-methylphenyl)cyclopentyl]carbonyl}amino)methyl]benzamide
N-hydroxy-4-({[3-(2-hydroxyphenyl)propanoyl]amino}methyl)benzamide
N-hydroxy-4-{[(4-phenylbutanoyl)amino]methyl}benzamide
N-hydroxy-4-({[4-(4-methylphenyl)butanoyl]amino}methyl)benzamide
4-({[(2E)-3-(2-furyl)prop-2-enoyl]amino}methyl)-N-hydroxybenzamide
N-{4-[(hydroxyamino)carbonyl]benzyl}-1H-indole-3-carboxamide
N-{4-[(hydroxyamino)carbonyl]benzyl}-1H-indole-2-carboxamide
N-hydroxy-4-({[(2E)-3-(4-methylphenyl)prop-2-enoyl]amino}methyl)benzamide
N-hydroxy-4-{[(2-hydroxy-3-nitrobenzyl)amino]methyl}benzamide
N-hydroxy-4-({[(4-methoxy-1-naphthyl)methyl]amino}methyl)benzamide
N-hydroxy-4-{[(2-nitrobenzyl)amino]methyl}benzamide
4-{[(biphenyl-2-ylmethyl)amino]methyl}-N-hydroxybenzamide
4-{[(2-fluoro-6-methoxybenzyl)amino]methyl}-N-hydroxybenzamide
N-hydroxy-4-{[(2-hydroxy-6-methoxybenzyl)amino]methyl}benzamide
N-hydroxy-4-({[(2-hydroxy-1-naphthyl)methyl]amino}methyl)benzamide
4-({[4-(benzyloxy)benzyl]amino}methyl)-N-hydroxybenzamide
4-{[(3-fluoro-4-methylbenzyl)amino]methyl}-N-hydroxybenzamide
N-hydroxy-4-{[(2-naphthylmethyl)amino]methyl}benzamide
N-hydroxy-4-[({[(2-hydroxy-6-methylphenyl)amino]carbonyl}amino)methyl]benzamide
N-hydroxy-4-[({[(2-hydroxy-4-nitrophenyl)amino]carbonyl}amino)methyl]benzamide
N-hydroxy-4-[({[(2-hydroxyphenyl)amino]carbonyl}amino)methyl]benzamide
N-hydroxy-4-[({[(3-hydroxy-2-naphthyl)amino]carbonyl}amino)methyl]benzamide
N-hydroxy-4-[({[(2-mercaptophenyl)amino]carbonyl}amino)methyl]benzamide
4-[({[(3-acetylphenyl)amino]carbonyl}amino)methyl]-N-hydroxybenzamide
N-hydroxy-4-[({[(2-nitropyridin-3-yl)amino]carbonyl}amino)methyl]benzamide
4-[({[(2-aminophenyl)amino]carbonyl}amino)methyl]-N-hydroxybenzamide
4-[({[(8-amino-1-naphthyl)amino]carbonyl}amino)methyl]-N-hydroxybenzamide;
methyl N⁵-hydroxy-N²-[2-(4-methylphenyl)propanoyl]glutaminate
ethyl N⁵-hydroxy-N²-[2-(4-methylphenyl)propanoyl]glutaminate
methyl N²-(biphenyl-4-ylacetyl)-N⁵-hydroxyglutaminate
ethyl N²-(biphenyl-4-ylacetyl)-N⁵-hydroxyglutaminate
methyl N⁵-hydroxy-N²-{[1-(4-methylphenyl)cyclopentyl]carbonyl}glutaminate
ethyl N⁵-hydroxy-N²-{[1-(4-methylphenyl)cyclopentyl]carbonyl}glutaminate
methyl N⁵-hydroxy-N²-[3-(2-hydroxyphenyl)propanoyl]glutaminate
ethyl N⁵-hydroxy-N²-[3-(2-hydroxyphenyl)propanoyl]glutaminate
methyl N⁵-hydroxy-N²-(4-phenylbutanoyl)glutaminate
ethyl N⁵-hydroxy-N²-(4-phenylbutanoyl)glutaminate
methyl N⁵-hydroxy-N²-[4-(4-methylphenyl)butanoyl]glutaminate
ethyl N⁵-hydroxy-N²-[4-(4-methylphenyl)butanoyl]glutaminate
methyl N²-[(2E)-3-(3-furyl)prop-2-enoyl]-N⁵-hydroxyglutaminate
ethyl N²-[(2E)-3-(2-furyl)prop-2-enoyl]-N⁵-hydroxyglutaminate
methyl N⁵-hydroxy-N²-(1H-indol-3-ylcarbonyl)glutaminate
ethyl N⁵-hydroxy-N²-(1H-indol-3-ylcarbonyl)glutaminate
methyl N⁵-hydroxy-N²-(1H-indol-2-ylcarbonyl)glutaminate
ethyl N⁵-hydroxy-N²-(1H-indol-2-ylcarbonyl)glutaminate
methyl N⁵-hydroxy-N²-[(2E)-3-(4-methylphenyl)prop-2-enoyl]glutaminate
ethyl N⁵-hydroxy-N²-[(2E)-3-(4-methylphenyl)prop-2-enoyl]glutaminate
1-(2-hydroxy-4-methylbenzoyl)prolinamide
N-hydroxy-1-(2-hydroxy-4-methylbenzoyl)prolinamide
1-(biphenyl-4-ylcarbonyl)prolinamide
1-(biphenyl-4-ylcarbonyl)-N-hydroxyprolinamide
1-(4-tert-butylbenzoyl)prolinamide
1-(4-tert-butylbenzoyl)-N-hydroxyprolinamide
1-(1-hydroxy-2-naphthoyl)prolinamide
N-hydroxy-1-(1-hydroxy-2-naphthoyl)prolinamide
1-(3-hydroxy-2-naphthoyl)prolinamide
N-hydroxy-1-(3-hydroxy-2-naphthoyl)prolinamide
1-(quinolin-6-ylcarbonyl)prolinamide
N-hydroxy-1-[(7-hydroxyquinolin-6-yl)carbonyl]prolinamide
1-(isoquinolin-3-ylcarbonyl)prolinamide
N-hydroxy-1-(isoquinolin-3-ylcarbonyl)prolinamide
1-(pyridin-2-ylcarbonyl)prolinamide
N-hydroxy-1-(pyridin-2-ylcarbonyl)prolinamide
1-[(2-mercaptopyridin-3-yl)carbonyl]prolinamide
N-hydroxy-1-[(2-mercaptopyridin-3-yl)carbonyl]prolinamide
1-(isoquinolin-1-ylcarbonyl)prolinamide
N-hydroxy-1-(isoquinolin-1-ylcarbonyl)prolinamide;
4-amino-N-benzyl-3-hydroxybenzamide
4-amino-3-hydroxy-N-(2-hydroxybenzyl)benzamide
4-amino-N-(4-fluorobenzyl)-3-hydroxybenzamide
4-amino-N-(3,5-dimethoxybenzyl)-3-hydroxybenzamide
4-amino-3-hydroxy-N-(2-naphthylmethyl)benzamide
4-amino-3-hydroxy-N-(2-phenoxyethyl)benzamide
4-amino-3-hydroxy-N-[1-(2-naphthyl)ethyl]benzamide
4-amino-3-hydroxy-N-(2-phenylethyl)benzamide
4-amino-3-hydroxy-N-(3-phenylpropyl)benzamide
4-amino-3-hydroxy-N-(4-phenylbutyl)benzamide;
5-amino-N-benzyl-2-hydroxybenzamide
5-amino-2-hydroxy-N-(2-hydroxybenzyl)benzamide
5-amino-N-(4-fluorobenzyl)-2-hydroxybenzamide
5-amino-N-(3,5-dimethoxybenzyl)-2-hydroxybenzamide
5-amino-2-hydroxy-N-(2-naphthylmethyl)benzamide
5-amino-2-hydroxy-N-(2-phenoxyethyl)benzamide
5-amino-2-hydroxy-N-[1-(2-naphthyl)ethyl]benzamide
5-amino-2-hydroxy-N-(2-phenylethyl)benzamide
5-amino-2-hydroxy-N-(3-phenylpropyl)benzamide and
5-amino-2-hydroxy-N-(4-phenylbutyl)benzamide.

The above names were generated using ChemDraw Ultra v. 9.0.1, which is available from CambridgeSoft.

### Example 4

SSAO activity determination: All assays were performed at 37° C. with SSAO from human or mice adipose tissue. The enzyme activity was measured through detection of hydrogen peroxide formed by the oxidation of benzylamine. This method is based on the horseradish peroxidase catalyzed hydrogen peroxide oxidation of 10-acetyl-3,7-dihydroxyphenoxazine (Molecular Probes A-6550), that produces resorufin a highly fluorescent product (excitation, 545 nm; emission, 590 nm) (Zhou and Panchuk-Voloshina, 1997). Human or mice adipose tissue homogenates, used as a source of SSAO activity, were preincubated in 96 well microplates for 20 min at 37° C. in 180 μL of 200 mM Phosphate buffer and H₂O₂-detecting mixture containing horseradish peroxidase (final concentration 1 U/mL) and Amplex Red reagent (60 μM) and different concentrations of inhibitors when necessary. Catalytic reaction was initiated by addition of 20 μL of benzylamine as substrate at 10 mM for human homogenates giving final concentrations of 100 μM and 1 mM respectively. Fluorescence intensity of the samples was measured continuously during 1 h (excitation, 545 nm; emission, 590 nm) and H₂O₂ concentration was calculated from calibration curves generated by serial dilutions of standard H₂O₂. To evaluate the amount of H₂O₂ formed specifically via SSAO-mediated reaction, semicarbazide 100 μM was included in the control wells subjected to the same treatments and these values were subtracted from the total amount of H₂O₂ formed.

The inhibition was measured as % decrease of the signal compared to a control without inhibitor. Blank values in absence of substrates were subtracted from the fluorescence for each experimental condition. The IC₅₀ shown in Table 1 were calculated with GraphPad Prism 4 program.

TABLE I

| | IC₅₀, μM on Human |
|---|---|
| Ref. N^{o} | SSAO |
| 1 | 0.39 ± 0.07 |
| 2 | 1.10 ± 0.04 |
| 3 | 0.10 ± 0.03 |
| 4 | 0.033 ± 0.006 |
| 5 | 0.25 ± 0.02 |
| 6 | 0.3 ± 0.1 |
| 7 | 1.9 ± 0.2 |
| 8 | 0.066 ± 0.001 |
| 9 | 0.136 ± 0.040 |
| 10 | 0.041 ± 0.008 |
| 11 | 0.0040 ± 0.0005 |
| 12 | 1.575 ± 0.175 |
| 13 | 0.067 ± 0.001 |
| 14 | 0.054 ± 0.007 |
| 15 | 0.217 ± 0.039 |
| 16 | 0.028 ± 0.008 |
| 17 | 0.217 ± 0.039 |
| 18 | 0.041 ± 0.001 |
| 19 | 0.140 ± 0.030 |
| 20 | 0.789 ± 0.061 |
| 21 | 0.097 ± 0.021 |
| 22 | 0.369 ± 0.071 |
| 23 | 0.70 ± 0.04 |
| 24 | 0.334 ± 0.080 |
| 25 | 0.941 ± 0.026 |
| 26 | 0.125 ± 0.002 |
| 27 | 0.214 ± 0.041 |
| 28 | 0.229 ± 0.092 |
| 29 | 0.259 ± 0.062 |
| 30 | 0.165 ± 0.007 |
| 31 | 0.128 ± 0.03 |
| 32 | 0.121 ± 0.047 |
| 33 | 0.090 ± 0.002 |
| 34 | 0.147 ± 0.044 |
| 35 | 0.160 ± 0.031 |
| 36 | 0.078 ± 0.024 |
| 37 | 0.18 ± 0.01 |
| 38 | 0.14 ± 0.01 |
| 39 | 0.39 ± 0.04 |
| 40 | 0.202 ± 0.026 |
| 41 | 0.25 ± 0.056 |
| 42 | 0.173 ± 0.009 |
| 43 | 0.337 ± 0.07 |
| 44 | 0.185 ± 0.031 |
| 45 | 0.146 ± 0.005 |
| 46 | 0.401 ± 0.030 |
| 47 | 0.41 ± 0.063 |
| 48 | 0.074 |
| 49 | 0.253 ± 0.002 |
| 50 | 0.577 ± 0.027 |
| 51 | 0.477 ± 0.064 |
| 52 | 0.189 ± 0.016 |
| 53 | 0.618 ± 0.118 |
| 54 | 0.575 ± 0.023 |
| 55 | 0.762 ± 0.060 |

It should be understood that the foregoing disclosure emphasizes certain specific embodiments of the invention and that all modifications or alternatives equivalent thereto are within the spirit and scope of the invention as set forth in the appended claims.

The invention and the manner and process of making and using it, are now described in such full, clear, concise and exact terms as to enable any person skilled in the art to which it pertains, to make and use the same. It is to be understood that the foregoing describes preferred embodiments of the invention and that modifications may be made therein without departing from the spirit or scope of the invention as set forth in the claims. To particularly point out and distinctly claim the subject matter regarded as invention, the following claims conclude this specification.

1-42. (canceled)

43. A compound of formula:
R₃—(Y)ₙ—(CR₂R₂)ₘ-Z
or a pharmaceutically acceptable salt thereof, wherein
m is 1-4;
n is 1;
Z is CONR₁₀H;
Y is —C(O)NH—;
R₁ is independently H or C₁-C₆ alkyl;
R₂ is independently H or C₁-C₆ alkyl, wherein the C₁-C₆ alkyl is optionally substituted with OH, NH₂, or SH; and
R₃ is aryl selected from phenyl, naphthyl, indanyl, and biphenyl, C₅-C₆ cycloalkyl, heteroaryl
selected from pyridyl, pyrimidyl, indolyl, pyrrolyl, thienyl, furanyl, thiazolyl, pyrazolyl,
and oxazolyl, heterocycloalkyl selected from piperazinyl, piperidinyl, pyrrolidinyl,
quinolinyl, tetrahydropyranyl, morpholinyl, thiomorpholinyl, and
S,S-dioxothiomorpholinyl, each of which is optionally substituted with 1, 2, 3, 4, or 5 groups that are independently C₁-C₆ alkyl, C₁-C₆ alkoxy, halogen, CF₃, OCF₃, nitro, CN, CO₂H, C₁-C₆ alkylthio, C₁-C₆ acyloxy, phenyl, pyridyl, thienyl, furanyl, pyrimidyl, or hydroxy.

44. The compound according to claim 43 that is N¹-hydroxy-N²-[(4-bromophenyl)carbonyl]glycinamide.

45. A compound of formula 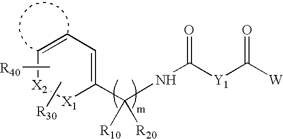
wherein
R₁₀ and R₂₀ are independently H, C₁-C₆ alkyl, C₂-C₆ alkenyl, C₂-C₆ alkynyl, aryl, substituted aryl, heterocycloalkyl containing one or two heteroatoms selected from S, N, and O, or C₃-C₇ cycloalkyl, and where any member of the alkyl, aryl, or cycloalkyl group is optionally substituted with halogen, C₁-C₆ alkyl or C₁-C₆ alkoxy, aryl or substituted aryl;
R₃₀ and R₄₀ are independently H, OH, SH, halogen, nitro, amino, mono or di(C₁-C₆ alkyl)amino, C₁-C₆ alkyl, C₁-C₆ alkanoyl, aryl, substituted aryl, C₁-C₆ alkoxy, C₁-C₆ alkoxyalkyl, C₃-C₇ cycloalkyl, C₃-C₇ cycloalkylalkyl, C₃-C₇ cycloalkylalkoxy, heteroaryl, heteroarylalkyl, heterocycloalkyl, or heterocycloalkylalkyl, where the cyclic portion, the alkyl portion or a combination thereof of each of the above is optionally substituted with 1, 2, 3, 4, or 5 groups that are independently halogen, C₁-C₆ alkyl, C₁-C₆ alkoxy, aryl, substituted aryl, halogen, nitro, nitroso, aldehyde, carboxylic acid, —C(O)NH₂, —C(O)NH(C₁-C₆ alkyl), —C(O)N(C₁-C₆ alkyl)(C₁-C₆ alkyl), amino, mono or di(C₁-C₆ alkyl)amino, C₁-C₆ alkoxycarbonyl, sulfate, imine, hydroxyl or nitrile;
Y₁ is (CH₂), or aryl;
m is 0, 1, 2, 3, or 4;
n is 0, 1, 2, 3, or 4;
W is NHOH, NH₂, NHR₁₀, OR₁₀, or NH—NHR₁₀;
X₁ and X₂ are independently C, CH, or N, provided that X₁ and X₂ are not simultaneously N;
where the dashed lines represent a cycloalkyl or aryl group that is optionally present; and
n and m independently an integer from 1 to 5.

46. The compounds according to claim 45 wherein m is 0.

47. The compounds according to claim 46 wherein R₃₀ and R₄₀ are independently H, OH, SH, halogen, nitro, amino, mono or di(C₁-C₆ alkyl)amino, C₁-C₆ alkyl, C₁-C₆ alkanoyl, or C₁-C₆ alkoxy.

48. The compounds according to claim 47 wherein Y₁ is (CH₂)ₙ, and n is 0, 1, or 2.

49. The compounds according to claim 48 wherein W is NHOH.

50. The compounds according to claim 45 selected from
N-hydroxy-N′-(1-methyl-3-phenylpropyl)succinamide;
N-hydroxy-N′-(2-hydroxyphenyl)succinamide; and
N¹-hydroxy-N4-(1-(naphthalen-2-yl)ethyl)succinamide.

51. A compound of formula 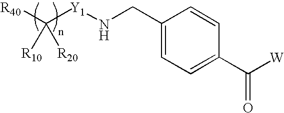
wherein
n is an integer from 1 to 5;
R₁₀ is independently H, C₁-C₆ alkyl, aryl, C₂-C₆ alkenyl, C₂-C₆alkynyl, substituted aryl, heterocycloalkyl containing one or two heteroatoms selected from S, N, and O, or C₃-C₇ cycloalkyl, and where any member of the alkyl, aryl, or cycloalkyl group is optionally substituted with halogen, C₁-C₆ alkyl, C₁-C₆ alkoxy, aryl, or substituted aryl;
R₂₀ is H, C₁-C₆ alkyl, aryl, substituted aryl, C₁-C₆ alkoxy, C₁-C₆ alkoxyalkyl, C₃-C₇ cycloalkyl, C₃-C₇ cycloalkylalkyl, C₃-C₇ cycloalkylalkoxy, heteroaryl, heteroarylalkyl, heterocycloalkyl, or heterocycloalkylalkyl, where each of the above is optionally substituted with 1, 2, 3, 4, or 5 groups that are independently halogen, C₁-C₆ alkyl, C₁-C₆ alkoxy, aryl, arylalkoxy, substituted aryl, halogen, nitro, nitroso, aldehyde, carboxylic acid, —C(O)NH₂, —C(O)NH(C₁-C₆ alkyl), —C(O)N(C₁-C₆ alkyl)(C₁-C₆ alkyl), amino, C₁-C₆ alkoxycarbonyl, sulfate, imine, hydroxyl or nitrile; or
R₁₀, R₂₀ and the carbon to which they are attached form a cycloalkyl ring;
R₄₀ is H, C₁-C₆ alkyl, aryl, substituted aryl, C₁-C₆ alkoxy, C₁-C₆ alkoxyalkyl, C₃-C₇ cycloalkyl, C₃-C₇ cycloalkylalkyl, C₃-C₇ cycloalkylalkoxy, heteroaryl, heteroarylalkyl, heterocycloalkyl, or heterocycloalkylalkyl, where each of the above is optionally substituted with 1, 2, 3, 4, or 5 groups that are independently halogen, C₁-C₆ alkyl, C₁-C₆ alkoxy, aryl, arylalkoxy, substituted aryl, halogen, nitro, nitroso, aldehyde, carboxylic acid, —C(O)NH₂, —C(O)NH(C₁-C₆ alkyl), —C(O)N(C₁-C₆ alkyl)(C₁-C₆ alkyl), amino, C₁-C₆ alkoxycarbonyl, sulfate, imine, hydroxyl or nitrile; or
Y₁ is SO₂, C(O), CH₂, or —NHC(O); and
W is NHOH, NHR₁₀, OR₁₀, or NH—NHR₁₀.

52. The compounds according to claim 51 wherein R₄₀ is phenyl, naphthyl, furanyl, indolyl, or quinolinyl, where each of the above is optionally substituted with 1, 2, or 3 groups that are independently halogen, C₁-C₆ alkyl, C₁-C₆ alkoxy, phenyl, halogen, nitro, carboxylic acid, —C(O)NH₂, —C(O)NH(C₁-C₆ alkyl), —C(O)N(C₁-C₆ alkyl)(C₁-C₆ alkyl), amino, C₁-C₆ alkoxycarbonyl, hydroxyl or nitrile.

53. The compounds according to claim 52 wherein n is 1 or 2 and R₁₀ and R₂₀ are both H.

54. The compounds according to claim 52 wherein R₁₀, R₂₀ and the carbon to which they are attached form a cycloalkyl ring and W is NHOH.

55. The compounds according to claim 51 selected from
N-hydroxy-4-[({[1-(4-methylphenyl)cyclopentyl]carbonyl}amino)methyl]benzamide;
and
N-hydroxy-4-({[3-(2-hydroxyphenyl)propanoyl]amino}methyl)benzamide.

56. A compound of formula 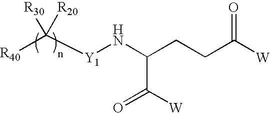
wherein
n is an integer from 1 to 5;
R₁₀ is independently H, C₁-C₆ alkyl, aryl, C₂-C₆ alkenyl, C₂-C₆alkynyl, substituted aryl,
heterocycloalkyl containing one or two heteroatoms selected from S, N, and O, or
C₃-C₇ cycloalkyl, and where any member of the alkyl, aryl, or cycloalkyl group is optionally substituted with halogen, C₁-C₆ alkyl or C₁-C₆ alkoxy, aryl or substituted aryl;
R₂₀ is independently H, C₁-C₆ alkyl, C₂-C₆ alkenyl, C₂-C₆ alkynyl, aryl, substituted aryl, heterocycloalkyl containing one or two heteroatoms selected from S, N, and O, or C₃-C₇ cycloalkyl, and where any member of the alkyl, aryl, or cycloalkyl group is optionally substituted with halogen, C₁-C₆ alkyl or C₁-C₆ alkoxy, aryl or substituted aryl;
R₃₀ is independently H, C₁-C₆ alkyl, aryl, substituted aryl, C₁-C₆ alkoxy, C₁-C₆ alkoxyalkyl, C₃-C₇ cycloalkyl, C₃-C₇ cycloalkylalkyl, C₃-C₇ cycloalkylalkoxy, heteroaryl, heteroarylalkyl, heterocycloalkyl, or heterocycloalkylalkyl, where each of the above is optionally substituted with 1, 2, 3, 4, or 5 groups that are independently halogen, C₁-C₆ alkyl, C₁-C₆ alkoxy, aryl, substituted aryl, halogen, nitro, nitroso, aldehyde, carboxylic acid, —C(O)NH₂, —C(O)NH(C₁-C₆ alkyl), —C(O)N(C₁-C₆ alkyl)(C₁-C₆ alkyl), amino, C₁-C₆ alkoxycarbonyl, sulfate, imine, hydroxyl or nitrile; or
R₂₀, R₃₀, and the carbon to which they are attached form a cycloalkyl ring; or 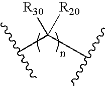
optionally encompasses olefins when n is 2-5, in such a case, R₂₀ is not present on the olefinic carbons.
R₄₀ is H, C₁-C₆ alkyl, aryl, substituted aryl, C₁-C₆ alkoxy, C₁-C₆ alkoxyalkyl, C₃-C₇ cycloalkyl, C₃-C₇ cycloalkylalkyl, C₃-C₇ cycloalkylalkoxy, heteroaryl, heteroarylalkyl, heterocycloalkyl, or heterocycloalkylalkyl, where each of the above is optionally substituted with 1, 2, 3, 4, or 5 groups that are independently halogen, C₁-C₆ alkyl, C₁-C₆ alkoxy, aryl, substituted aryl, halogen, nitro, nitroso, aldehyde, carboxylic acid, —C(O)NH₂, —C(O)NH(C₁-C₆ alkyl), —C(O)N(C₁-C₆ alkyl)(C₁-C₆ alkyl), amino, C₁-C₆ alkoxycarbonyl, sulfate, imine, hydroxyl or nitrile;
Y₁ is CH₂, C(O), or SO₂; and
W at each occurrence is independently NHOH, NHR₁₀, OR₁₀, or NH—NHR₁₀.

57. The compounds according to claim 56 wherein Y₁ is C(O).

58. The compounds according to claim 57 wherein R₄₀ is phenyl, indolyl, or furanyl, where each of the above is optionally substituted with 1, 2, or 3 groups that are independently halogen, C₁-C₆ alkyl, C₁-C₆ alkoxy, aryl, halogen, nitro, carboxylic acid, —C(O)NH₂, —C(O)NH(C₁-C₆ alkyl), —C(O)N(C₁-C₆ alkyl)(C₁-C₆ alkyl), amino, C₁-C₆ alkoxycarbonyl, hydroxyl or nitrile.

59. The compounds according to claim 57 wherein when n is 2; 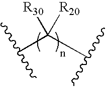
is a C₂ olefin; R₂₀ is absent and R₃₀ is H or C₁-C₄ alkyl.

60. The compounds according to claim 57 wherein R₂₀, R₃₀, and the carbon to which they are attached form a cycloalkyl ring and at least one W is OH.

61. The compounds according to claim 56 selected from
N-(biphenyl-4-ylacetyl)glutamic acid;
N⁵-hydroxy-N²-{[1-(4-methylphenyl)cyclopentyl]carbonyl}glutamine;
N-(4-phenylbutanoyl)glutamic acid;
N-[4-(4-methylphenyl)butanoyl]glutamic acid; and
N-[(2E)-3-(4-methylphenyl)prop-2-enoyl]glutamic acid.

62. A compound of formula 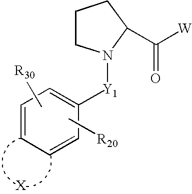
wherein
R₂₀ and R₃₀ are independently H, C₁-C₆ alkyl, aryl, substituted aryl, heterocycloalkyl containing one or two heteroatoms selected from S, N, and O, or C₃-C₇ cycloalkyl, and where any member of the alkyl, aryl, or cycloalkyl group is optionally substituted with halogen, OH, SH, C₁-C₆ alkyl or C₁-C₆ alkoxy, aryl, or substituted aryl;
W is NHOH, NHR₁₀, OR₁₀, or NH—NHR₁₀;
X is C, CH, or any heteroatom selected from S, N, and O;
Y₁ is CO, or CH₂; and
the dashed lines represent an optional aryl, heteroaryl or heterocycloalkyl ring.

63. The compounds according to claim 62 wherein Y₁ is CO.

64. The compounds according to claim 63 wherein 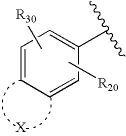
is phenyl, naphthyl, pyridyl, or quinolinyl, each of which is optionally substituted with 1, 2, or 3 groups that are independently OH, SH, C₁-C₄ alkyl, C₁-C₄ alkoxy, or phenyl.

65. The compounds according to claim 62 wherein W is NHOH or NH₂.

66. The compounds according to claim 62 selected from
1-(biphenyl-4-ylcarbonyl)prolinamide;
1-(4-tert-butylbenzoyl)prolinamide;
1-(3-hydroxy-2-naphthoyl)prolinamide; and
1-(quinolin-6-ylcarbonyl)prolinamide.

67. A compound of formula 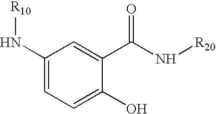
wherein
R₁₀ and R₂₀ are independently H, C₁-C₆ alkyl, C₂-C₆ alkenyl, C₂-C₆ alkynyl, aryl, substituted aryl, heterocycloalkyl containing one or two heteroatoms selected from S, N, and O, or C₃-C₇ cycloalkyl, and where any member of the alkyl, aryl, or cycloalkyl group is optionally substituted with halogen, C₁-C₆ alkyl or C₁-C₆ alkoxy, aryl or substituted aryl.

68. The compounds according to claim 67 wherein R₁₀ is H or C₁-C₄ alkyl.

69. The compounds according to claim 68 wherein R₂₀ is C₁-C₆ alkyl substituted with phenyl or naphthyl, where each is optionally substituted with 1 or 2 groups that are independently OH, halogen, C₁-C₄ alkyl, or C₁-C₄ alkoxy.

70. The compounds according to claim 67 selected from
5-amino-N-benzyl-2-hydroxybenzamide;
5-amino-2-hydroxy-N-(2-hydroxybenzyl)benzamide;
5-amino-N-(4-fluorobenzyl)-2-hydroxybenzamide;
5-amino-N-(3,5-dimethoxybenzyl)-2-hydroxybenzamide;
5-amino-2-hydroxy-N-(2-naphthylmethyl)benzamide;
5-amino-2-hydroxy-N-(2-phenylethyl)benzamide;
5-amino-2-hydroxy-N-(3-phenylpropyl)benzamide; and
5-amino-2-hydroxy-N-(4-phenylbutyl)benzamide.

71. A compound of formula 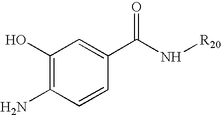
wherein
R₂₀ is —CH₂—X, —CH₂CH₂—X, —CH(CH₃)—X, —CH₂CH₂CH₂—X, —CH₂CH₂CH₂CH₂—X or —CH₂CH₂—O—X; and
X is phenyl or naphthyl optionally substituted with 1 or 2 groups selected from methoxy, —F, or —OH.

72. The compounds according to claim 71 selected from
4-amino-N-(4-fluorobenzyl)-3-hydroxybenzamide;
4-amino-3-hydroxy-N-(2-naphthylmethyl)benzamide;
4-amino-3-hydroxy-N-(2-phenoxyethyl)benzamide;
4-amino-3-hydroxy-N-[1-(2-naphthyl)ethyl]benzamide;
4-amino-3-hydroxy-N-(2-phenylethyl)benzamide;
4-amino-3-hydroxy-N-(3-phenylpropyl)benzamide; and
4-amino-3-hydroxy-N-(4-phenylbutyl)benzamide.

73. The compound N-hydroxy-2-(2-(2-methyl-1H-indol-3-yl)acetamido)acetamide.

74. The compound N-hydroxy-4-({[(4-methylphenyl)sulfonyl]amino}methyl)benzamide.

75. The compound N-(1H-indol-2-ylcarbonyl)glutamic acid.

76. The compound 1-(isoquinolin-1-ylcarbonyl)prolinamide.

77. The compound 5-amino-2-hydroxy-N-(2-phenoxyethyl)benzamide.

78. The compound selected from
N²-(biphenyl-4-ylsulfonyl)-N¹-hydroxythreoninamide;
3-amino-N²-(biphenyl-4-ylsulfonyl)-N¹-hydroxyalaninamide;
N¹-hydroxy-N²-[(4-propylphenyl)sulfonyl]serinamide;
N¹-hydroxy-N²-[(4-propylphenyl)sulfonyl]threoninamide;
N²-{[4-(1,1-dimethylpropyl)phenyl]sulfonyl}-N¹-hydroxyserinamide;
N²-{[4-(1,1-dimethylpropyl)phenyl]sulfonyl}-N¹-hydroxythreoninamide;
N²-{[4-(1,1-dimethylpropyl)phenyl]sulfonyl}-N¹-hydroxyglycinamide;
N¹-hydroxy-N²-(2-naphthylsulfonyl)serinamide;
N¹-hydroxy-N²-(2-naphthylsulfonyl)threoninamide;
N¹-hydroxy-N²-[(4-methyl-3-nitrophenyl)sulfonyl]serinamide;
N¹-hydroxy-N²-(2-naphthylsulfonyl)glycinamide;
N¹-hydroxy-N²-[(4-methylphenyl)sulfonyl]threoninamide;
3-amino-N¹-hydroxy-N²-[(4-methylphenyl)sulfonyl]alaninamide;
N¹-hydroxy-N²-(quinolin-7-ylsulfonyl)threoninamide;
N²-{[8-(dimethylamino)-2-naphthyl]sulfonyl}-N¹-hydroxyserinamide; and
N²-{[8-(dimethylamino)-2-naphthyl]sulfonyl}-N¹-hydroxythreoninamide.

79. A method of treating an animal having a disease or disorder characterized by pathological expression of a copper-containing amine oxidase enzyme, comprising administering to an animal in need of such treatment an inhibitor of said copper-containing amine oxidase at a therapeutically-active concentration, wherein the inhibitor is a compound of formula
R₃—(Y)ₙ—(CR₂R₂)ₘ-Z
or a pharmaceutically acceptable salt thereof, wherein
m is 0 or 1-6;
n is 0 or 1;
Z is CONR₁₀H, COOH, B(OH)₂, SO₂NR₁OH, OR₁, SR₁, NHR₁, PO₃H, CH₂NHR₁, COR₁, CONHR₁, CHNR₁, or CNR₁NHR₁;
Y at each occurrence is independently —CO—, —CS—, —NR₂OR₂—, —NR₂—, —SR₂—, —NR₂SO₂R₂—, —COR₂—, —NR₂—C(NR₂)—NR₂—, —(C₁-C₆ alkyl)-NHC(O)—, —(C₁-C₆ alkyl)-N(C₁-C₆ alkyl)C(O)—, —NHC(O)—(C₁-C₆ alkyl)-, —N(C₁-C₆ alkyl)C(O)—(C₁-C₆ alkyl)-, —NHC(O)—, —N(C₁-C₆alkyl)C(O)—, —C(O)NH—, —C(O)—N(C₁-C₆alkyl), —SO₂NH—, —SO₂—N(C₁-C₆alkyl)-, —(C₁-C₆ alkyl)-C(O)NH—, —(C₁-C₆ alkyl)-C(O)—N(C₁-C₆alkyl)-, —O—(C₁-C₆ alkyl)-NHC(O)—, or —O—(C₁-C₆ alkyl)-N(C₁-C₆ alkyl)C(O)—, wherein the alkyl portion or portions of each of the above is optionally substituted with 1, 2, or 3 groups that are independently halogen, C₁-C₄ alkoxy, amino, mono or di (C₁-C₆ alkyl)amino, OH, indolyl, pyrrolyl, pyridyl, furanyl, guanidinyl, carboxyl, or ═O;
R₁ at each occurrence is independently H, C₁-C₆ alkyl, aryl, substituted aryl, heterocycloalkyl containing at least one and no more than two heteroatoms selected from S, N, and O, or C₃-C₇ cycloalkyl, and where any member of the alkyl, aryl/or cycloalkyl group is optionally substituted with halogen, C₁-C₆ alkyl or C₁-C₆ alkoxy, aryl or substituted aryl;
R₂ at each occurrence is independently H, C₁-C₆ alkyl, carboxyl, C₁-C₆ alkoxycarbonyl, aryl, substituted aryl, C₁-C₆ alkoxy, C₁-C₆ alkoxyalkyl, C₃-C₇ cycloalkyl, C₃-C₇ cycloalkylalkyl, C₃-C₇ cycloalkylalkoxy, heteroaryl, heteroarylalkyl, heterocycloalkyl, or heterocycloalkylalkyl, where each of the above is optionally substituted with 1, 2, 3, 4, or 5 groups that are independently halogen, C₁-C₆ alkyl, C₁-C₆ alkoxy, aryl, substituted aryl, halogen, nitro, nitroso, aldehyde (CHO), carboxylic acid, —C(O)NH₂, —C(O)NH(C₁-C₆ alkyl), —C(O)N(C₁-C₆ alkyl)(C₁-C₆ alkyl), amino, NH(C₁-C₆ alkyl), N(C₁-C₆ alkyl)(C₁-C₆ alkyl), C₁-C₆ alkoxycarbonyl, sulfate, imine, hydroxyl, —SH, or nitrile; and R₃ is aryl, C₁-C₆ alkyl, C₂-C₄ alkenyl, C₃-C₇ cycloalkyl, C₃-C₇ cycloalkyl C₁-C₆ alkyl, C₃-C₇ cycloalkyl C₁-C₆ alkoxy, heteroaryl, heterocycloalkyl, each of which is optionally substituted with 1, 2, 3, 4, or 5 groups that are independently C₁-C₆ alkyl, C₁-C₆ alkoxy, halogen, haloalkyl, haloalkoxy, nitro, amino, NH(C₁-C₆ alkyl), N(C₁-C₆ alkyl)(C₁-C₆ alkyl), CN, CO₂H, C₁-C₆ alkylthio, C₁-C₄ haloalkyl, C₁-C₄ haloalkoxy, C₁-C₆ acyloxy, aryl, heteroaryl, or hydroxyl, where the aryl and heteroaryl substituents on R₃ are further optionally substituted with one or more groups that are independently C₁-C₆ alkyl, amino, NH(C₁-C₆ alkyl), N(C₁-C₆ alkyl)(C₁-C₆ alkyl), OH, NO₂, C₁-C₆ alkoxy, halogen, arylalkoxy (in one aspect, phenylalkoxy, in another aspect, phenylCl alkoxy), haloalkyl (in one aspect, CF₃), haloalkoxy (in one aspect, OCF₃), thiol, or C₂-C₆ alkanoyl (in one aspect, C₂ alkanoyl); at a therapeutically-active concentration.

80. A method according to claim 79, wherein the copper-containing amine oxidase is semicarbazide-sensitive amine oxidase.

81. A method according to claim 79, wherein the animal is a human.

82. A method according to claim 79, wherein the disease or disorder is an inflammatory disease, an adipocyte dysfunction related disease, a carbohydrate metabolism related disease, a vascular disease, a neurodegenerative disease or cancer.

83. A method according to claim 82, wherein said inflammatory disease is rheumatoid arthritis, osteoarthritis, spondylitis, bone resorption disease, sepsis, septic shock, chronic pulmonary inflammatory disease, fever, periodontal diseases, ulcerative colitis, pyresis, cystic fibrosis, dysfunctions of the immune system, multiple sclerosis, inflammatory eye conditions including uveitis, glaucoma or conjunctivitis.

84. A method according to claim 82, wherein said inflammatory disease is degenerative bone or joint conditions including osteoarthritis, rheumatoid arthritis, rheumatoid spondylitis, gouty arthritis ankylosing spondylitis, psoriatic arthritis and other arthritic conditions, or joint inflammation.

85. A method according to claim 82, wherein said inflammatory disease is a chronic inflammatory skin condition, that is allergic lesions, lichen planus, pityriasis rosea, eczema, psoriasis, or dermatitis.

86. A method according to claim 82, wherein said inflammatory disease is a disease or disorder of the gastrointestinal tract, that is inflammatory bowel disease, Crohn's disease, atrophic gastritis, gastritis varialoforme, ulcerative colitis, coeliac disease, regional ileitis, peptic ulceration, particularly irritable bowel syndrome, reflux oesophagitis, and damage to the gastrointestinal tract resulting from infections by Helicobacter pylori or other infectious organism or from treatments with non-steroidal anti-inflammatory drugs;

87. A method according to claim 82, wherein said inflammatory disease is an inflammatory lung disorder that is asthma, bronchitis, particularly chronic obstructive pulmonary disease, farmer's lung, or acute respiratory distress syndrome.

88. A method according to claim 82, wherein said inflammatory disease is meningitis, pancreatitis, bacteraemia, endotoxaemia (septic shock), aphthous ulcers, gingivitis, pyresis, or pain, comprising inflammatory pain, neuropathic pain, acute pain or pain of a central origin.

89. A method according to claim 82, wherein said inflammatory disease is a central nervous system inflammatory condition or disease comprising multiple sclerosis, Alzheimer's disease, or ischaemia-reperfusion injury associated with ischemic stroke.

90. A method according to claim 82, wherein the carbohydrate metabolism related disease is diabetes or a complication of diabetes that is a microvascular or macrovascular disease comprising atherosclerosis, vascular retinopathies, nephropathies, neuropathies, joint problems or foot ulcers.

91. A method according to claim 82, wherein the adipocyte metabolism dysfunction is obesity or complications thereof comprising diabetes, hypertension or atherosclerosis.

92. A method according to claim 82, wherein the neurodegenerative disease is Alzheime's disease or Parkinson's disease.

93. A method according to claim 82, wherein the vascular disease is atheromatous and nonatheromatous arteriosclerosis, ischemic heart disease, or Raynaud's Disease and Phenomenon.

94. A method according to claim 79, wherein the disease or disorder is atherosclerosis, a neurodegenerative disease, obesity, hypertension or cancer.

95. A pharmaceutical composition comprising a compound according to claim 43, or a pharmaceutically-acceptable salt thereof and a pharmaceutically-acceptable excipient, diluent or adjuvant thereof.

96. A pharmaceutical composition according to claim 95, wherein the compound is:
N¹-hydroxy-N²-[(4-bromophenyl)carbonyl]glycinamide.

97. A pharmaceutical composition comprising a compound according to claim 45, or a pharmaceutically-acceptable salt thereof and a pharmaceutically-acceptable excipient, diluent or adjuvant thereof.

98. A pharmaceutical composition according to claim 97, wherein the compound is:
N-hydroxy-N′-(1-methyl-3-phenylpropyl)succinamide;
N-hydroxy-N′-(2-hydroxyphenyl)succinamide; and
N¹-hydroxy-N4-(1-(naphthalen-2-yl)ethyl)succinamide.

99. A pharmaceutical composition comprising a compound according to claim 51, or a pharmaceutically-acceptable salt thereof and a pharmaceutically-acceptable excipient, diluent or adjuvant thereof.

100. A pharmaceutical composition according to claim 99, wherein the compound is:
N-hydroxy-4-[({[1-(4-methylphenyl)cyclopentyl]carbonyl}amino)methyl]benzamide;
and
N-hydroxy-4-({[3-(2-hydroxyphenyl)propanoyl]amino}methyl)benzamide.

101. A pharmaceutical composition comprising a compound according to claim 56, or a pharmaceutically-acceptable salt thereof and a pharmaceutically-acceptable excipient, diluent or adjuvant thereof.

102. A pharmaceutical composition according to claim 101, wherein the compound is:
N-(biphenyl-4-ylacetyl)glutamic acid;
N⁵-hydroxy-N²-{[1-(4-methylphenyl)cyclopentyl]carbonyl}glutamine;
N-(4-phenylbutanoyl)glutamic acid;
N-[4-(4-methylphenyl)butanoyl]glutamic acid; and
N-[(2E)-3-(4-methylphenyl)prop-2-enoyl]glutamic acid.

103. A pharmaceutical composition comprising a compound according to claim 62, or a pharmaceutically-acceptable salt thereof and a pharmaceutically-acceptable excipient, diluent or adjuvant thereof.

104. A pharmaceutical composition according to claim 103, wherein the compound is:
1-(biphenyl-4-ylcarbonyl)prolinamide;
1-(4-tert-butylbenzoyl)prolinamide;
1-(3-hydroxy-2-naphthoyl)prolinamide; and
1-(quinolin-6-ylcarbonyl)prolinamide.

105. A pharmaceutical composition comprising a compound according to claim 67, or a pharmaceutically-acceptable salt thereof and a pharmaceutically-acceptable excipient, diluent or adjuvant thereof.

106. A pharmaceutical composition according to claim 105, wherein the compound is:
5-amino-N-benzyl-2-hydroxybenzamide;
5-amino-2-hydroxy-N-(2-hydroxybenzyl)benzamide;
5-amino-N-(4-fluorobenzyl)-2-hydroxybenzamide;
5-amino-N-(3,5-dimethoxybenzyl)-2-hydroxybenzamide;
5-amino-2-hydroxy-N-(2-naphthylmethyl)benzamide;
5-amino-2-hydroxy-N-(2-phenylethyl)benzamide;
5-amino-2-hydroxy-N-(3-phenylpropyl)benzamide; and
5-amino-2-hydroxy-N-(4-phenylbutyl)benzamide.

107. A pharmaceutical composition comprising a compound according to claim 71, or a pharmaceutically-acceptable salt thereof and a pharmaceutically-acceptable excipient, diluent or adjuvant thereof.

108. A pharmaceutical composition according to claim 107, wherein the compound is:
4-amino-N-(4-fluorobenzyl)-3-hydroxybenzamide;
4-amino-3-hydroxy-N-(2-naphthylmethyl)benzamide;
4-amino-3-hydroxy-N-(2-phenoxyethyl)benzamide;
4-amino-3-hydroxy-N-[1-(2-naphthyl)ethyl]benzamide;
4-amino-3-hydroxy-N-(2-phenylethyl)benzamide;
4-amino-3-hydroxy-N-(3-phenylpropyl)benzamide; and
4-amino-3-hydroxy-N-(4-phenylbutyl)benzamide.

109. A pharmaceutical composition comprising a compound, or a pharmaceutically-acceptable salt thereof, and a pharmaceutically-acceptable excipient, diluent or adjuvant thereof, wherein the compound is selected from:
N-hydroxy-2-(2-(2-methyl-1H-indol-3-yl)acetamido)acetamide;
N-hydroxy-4-({[(4-methylphenyl)sulfonyl]amino}methyl)benzamide;
N-(1H-indol-2-ylcarbonyl)glutamic acid;
1-(isoquinolin-1-ylcarbonyl)prolinamide;
5-amino-2-hydroxy-N-(2-phenoxyethyl)benzamide;
N²-(biphenyl-4-ylsulfonyl)-N¹-hydroxythreoninamide;
3-amino-N²-(biphenyl-4-ylsulfonyl)-N¹-hydroxyalaninamide;
N¹-hydroxy-N²-[(4-propylphenyl)sulfonyl]serinamide;
N¹-hydroxy-N²-[(4-propylphenyl)sulfonyl]threoninamide;
N²-{[4-(1,1-dimethylpropyl)phenyl]sulfonyl}-N¹-hydroxyserinamide;
N²-{[4-(1,1-dimethylpropyl)phenyl]sulfonyl}-N-hydroxythreoninamide;
N²-{[4-(1,1-dimethylpropyl)phenyl]sulfonyl}-N¹-hydroxyglycinamide;
N¹-hydroxy-N²-(2-naphthylsulfonyl)serinamide;
N¹-hydroxy-N²-(2-naphthylsulfonyl)threoninamide;
N¹-hydroxy-N²-[(4-methyl-3-nitrophenyl)sulfonyl]serinamide;
N¹-hydroxy-N²-(2-naphthylsulfonyl)glycinamide;
N¹-hydroxy-N²-[(4-methylphenyl)sulfonyl]threoninamide;
3-amino-hydroxy-N²-[(4-methylphenyl)sulfonyl]alaninamide;
N¹-hydroxy-N²-(quinolin-7-yl sulfonyl)threoninamide;
N²-{[8-(dimethylamino)-2-naphthyl]sulfonyl}-N¹-hydroxyserinamide; and
N²-{[8-(dimethylamino)-2-naphthyl]sulfonyl}-N¹-hydroxythreoninamide.
